(19) United States
(12) Patent Application Publication
(10) Pub. No.: US 2017/0210741 A1
(43) Pub. Date: Jul. 27, 2017

(54) Benzimidazole Compounds, Use As Inhibitors of WNT Signaling Pathway in Cancers, and Methods for Preparation Thereof

(71) Applicants: Southern Research Institute, Birmingham, AL (US); UAB Research Foundation, Birmingham, AL (US)

(72) Inventors: Corinne E. AUGELLI-SZAFRAN, Homewood, AL (US); Mark J. Suto, Homewood, AL (US); Vibha Pathak, Hoover, AL (US); Han-Xun Wei, Birmingham, AL (US); Donald Buchsbaum, Alabaster, AL (US)

(21) Appl. No.: 15/417,956

(22) Filed: Jan. 27, 2017

Related U.S. Application Data
(60) Provisional application No. 62/287,895, filed on Jan. 27, 2016.

(51) Int. Cl. C07D 471/04; C07D 401/10; C07D 403/10; C07D 235/18

(52) U.S. Cl. CPC C07D 471/04; C07D 235/18; C07D 401/10; C07D 403/10

(57) ABSTRACT

The present disclosure is concerned with benzimidazole compounds that are capable of inhibiting Wnt signaling and methods of treating disease states such as, for example, cancer, using these compounds. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/287,895, filed on Jan. 27, 2016, which is incorporated herein by reference in its entirety.

### BACKGROUND

Wnt/β-catenin signaling plays an important role in embryonic development and can lead to tumor formation when aberrantly activated (Clevers and Nusse (2012) Wnt/beta-catenin signaling and disease. Cell 149, 1192-1205; Anastas and Moon (2013) WNT signaling pathways as therapeutic targets in cancer. Nature reviews. Cancer 13, 11-26; Kahn, M. (2014) Can we safely target the WNT pathway? Nature reviews. Drug discovery 13, 513-532). β-Catenin is the central player in signal transduction of this pathway. In the absence of Wnt ligands, β-catenin levels are efficiently regulated by a supramolecular complex containing adenomatous polyposis coli (APC), axin, and glycogen synthetase kinase 3β (GSK3β). This complex promotes β-catenin phosphorylation and subsequent β-catenin degradation. The action of this complex is inhibited upon the binding of Wnt to its receptors on the cell surface. As a result, β-catenin protein is stabilized and then enters the nucleus to form a complex with transcription factors of the T-cell factor/lymphoid enhancing factor (TCF/LEF) family to activate transcription of Wnt target genes that regulate cancer progression, including tumor initiation, tumor growth, cell senescence, cell death, differentiation, and metastasis ((Clevers and Nusse (2012) Wnt/beta-catenin signaling and disease. Cell 149, 1192-1205; Anastas and Moon (2013) WNT signaling pathways as therapeutic targets in cancer. Nature reviews. Cancer 13, 11-26; Kahn, M. (2014) Can we safely target the WNT pathway? Nature reviews. Drug discovery 13, 513-532).

Several components of the Wnt/β-catenin pathway have been identified as oncogenes or tumor suppressors (showing gain-of-function or loss-of-function mutations, respectively) in colorectal cancer (Clevers and Nusse (2012) Wnt/beta-catenin signaling and disease. Cell 149, 1192-1205; Anastas and Moon (2013) WNT signaling pathways as therapeutic targets in cancer. Nature reviews. Cancer 13, 11-26; Kahn, M. (2014) Can we safely target the WNT pathway? Nature reviews. Drug discovery 13, 513-532). About 80% of all colorectal cancers contain loss-of function mutations in the tumor suppressor gene APC. Gain-of function mutations in the oncogene CTNNB1 (β-catenin encoding gene) are present in approximately 10% of the colorectal cancers. The consequence of either APC inactivation or CTNNB1 activation mutation is the failure of proper β-catenin degradation leading to its cytosolic accumulation. The β-catenin then translocates into the nucleus where it interacts with TCF/LEF to induce the expression of downstream target genes (Kinzler and Vogelstein (1996) Lessons from hereditary colorectal cancer. Cell 87, 159-170; Morin et al. (1997) Activation of beta-catenin-Tcf signaling in colon cancer by mutations in beta-catenin or APC. Science 275, 1787-1790). It is well established that aberrant activation of Wnt/β-catenin signaling is a necessary initiating event in the genesis of most colorectal cancer (Kinzler and Vogelstein (1996) Lessons from hereditary colorectal cancer. Cell 87, 159-170; Morin et al. (1997) Activation of beta-catenin-Tcf signaling in colon cancer by mutations in beta-catenin or APC. Science 275, 1787-1790; Dow et al. (2015) Apc Restoration Promotes Cellular Differentiation and Reestablishes Crypt Homeostasis in Colorectal Cancer. Cell 161, 1539-1552), and that the Wnt/β-catenin pathway has emerged as one of the most promising targets for colorectal cancer chemoprevention and treatment (Anastas and Moon (2013) WNT signaling pathways as therapeutic targets in cancer. Nature reviews. Cancer 13, 11-26; Kahn, M. (2014) Can we safely target the WNT pathway? Nature reviews. Drug discovery 13, 513-532; de Sousa, E. M., Vermeulen, L., Richel, D., and Medema, J. P. (2011). Targeting Wnt signaling in colon cancer stem cells. Clinical cancer research: an official journal of the American Association for Cancer Research 17, 647-653.]

While genetic mutations of APC and CTNNB1 are significant contributing factors for colorectal cancers, they are typically not the predominate mechanism associated with many other types of cancer such as breast, prostate, ovarian, and pancreatic cancers. Instead, it appears that dysregulation of Wnt/β-catenin signaling on the cell surface leads to aberrant activation of this pathway in these types of cancer (King et al. (2012) The Wnt/beta-catenin signaling pathway: a potential therapeutic target in the treatment of triple negative breast cancer. Journal of cellular biochemistry 113, 13-18; King et al. (2012) Frizzled7 as an emerging target for cancer therapy. Cellular signaling 24, 846-851; Arend et al. (2013) The Wnt/beta-catenin pathway in ovarian cancer: a review. Gynecologic oncology 131, 772-779; Verras and Sun (2006) Roles and regulation of Wnt signaling and beta-catenin in prostate cancer. Cancer letters 237, 22-32; Morris et al. (2010) KRAS, Hedgehog, Wnt and the twisted developmental biology of pancreatic ductal adenocarcinoma. Nature reviews. Cancer 10, 683-695). For example, studies have demonstrated that the Wnt/β-catenin signaling pathway is preferentially activated in triple negative breast cancer (TNBC), and that Wnt-co-receptor LRP6 is up-regulated in human TNBC (Lindvall et al. (2009) The Wnt co-receptor Lrp6 is required for normal mouse mammary gland development. PloS one 4, e5813; Liu et al. (2010) LRP6 overexpression defines a class of breast cancer subtype and is a target for therapy. Proceedings of the National Academy of Sciences of the United States of America 107, 5136-5141; Yang et al. (2011) FZD7 has a critical role in cell proliferation in triple negative breast cancer. Oncogene 30, 4437-4446). LRP6 expression is also significantly up-regulated in prostate patients with metastatic disease compared to those without metastasis, and is associated with a significantly increased risk of recurrent disease (Liu et al. (2012) N-myc downstream regulated gene 1 modulates Wnt-beta-catenin signaling and pleiotropically suppresses metastasis. EMBO molecular medicine 4, 93-108). Furthermore, recent studies indicated that among the Frizzled family, Frizzled 7 (Fzd7) is the Wnt receptor most commonly upregulated in a variety of cancers including colorectal cancer, hepatocellular carcinoma, and TNBC, and that Fzd7 plays an important role in stem cell biology and cancer development and progression (King et al. (2012) Frizzled7 as an emerging target for cancer therapy. Cellular signaling 24, 846-851).

Despite research indicating that targeted inhibition of Wnt/β-catenin signaling at the cell surface may provide a promising approach for cancer therapy, the development of potent and selective Wnt selective has remained elusive. Thus, there remains a need for small molecule inhibitors of Wnt.

### SUMMARY

In accordance with the purpose(s) of the invention, as embodied and broadly described herein, the invention, in one aspect, relates to benzimidazole compounds that inhibit Wnt signaling, and therefore find utility in the treatment of a number of disorders including, but not limited to, cancers such as pancreatic cancer, breast cancer, and colon cancer.

The present disclosure relates to compounds having a structure represented by a formula:

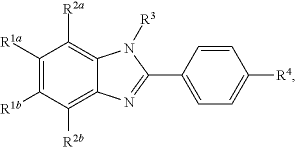

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

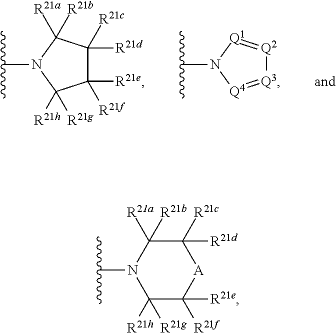

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; wherein each of Q¹, Q², Q³, and Q⁴, when present, is independently selected from CR³⁰ and N; and wherein each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are compounds having a structure represented by a formula:

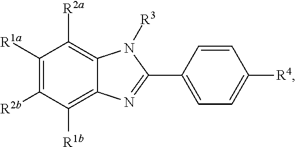

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

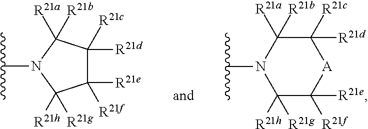

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; and wherein each of R^{21a}, R^{21b}, R^{21c}, R^{21d}, R^{21e}, R^{21f}, R^{21g}, and R^{21h} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are compounds having a structure selected from:

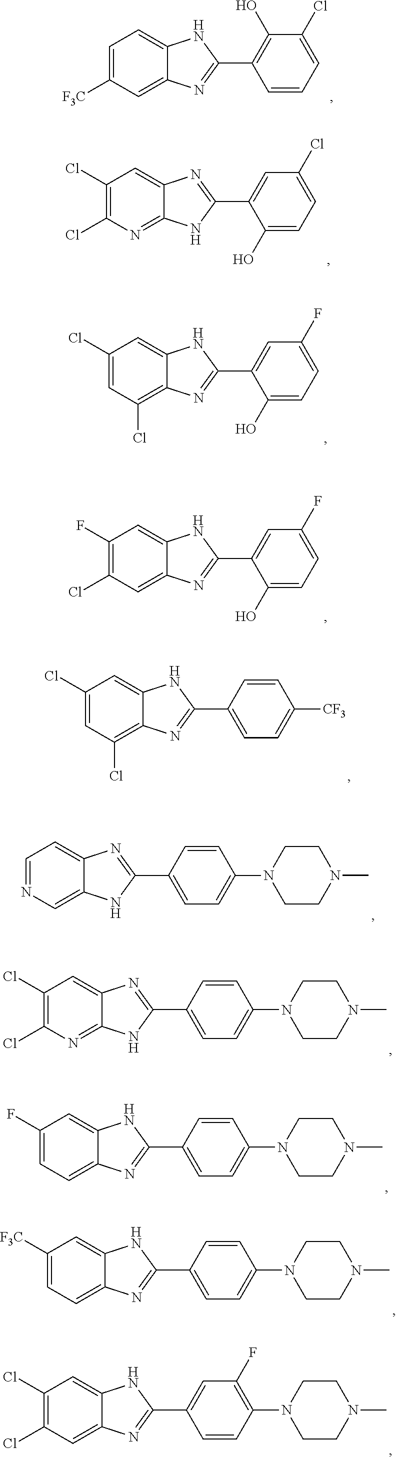

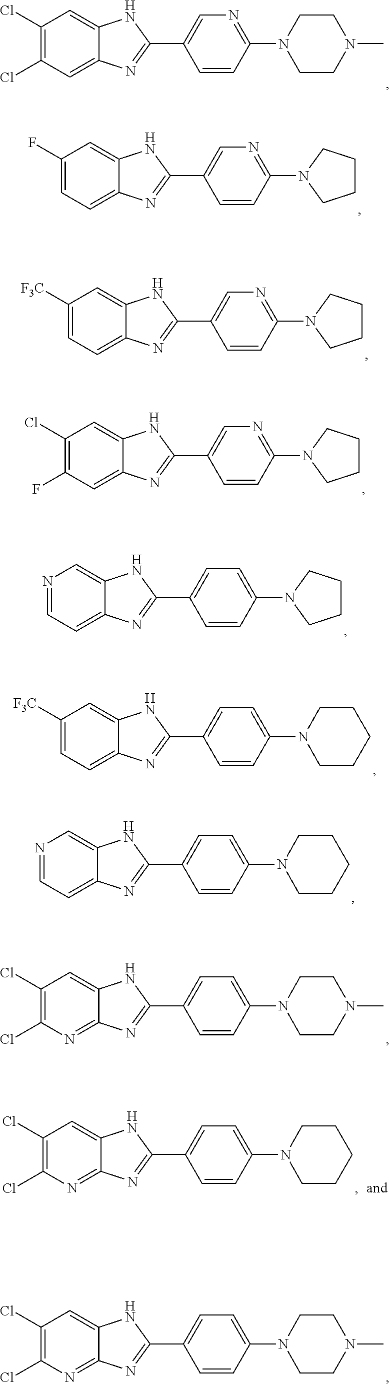

or a pharmaceutically acceptable salt thereof.

Also disclosed are methods of treating cancer in a subject, the method comprising administering to the subject an effective amount of at least one disclosed compound, or a pharmaceutically acceptable salt thereof.

Also disclosed are methods of treating cancer in a subject, the method comprising administering to the subject an effective amount of at least one compound having a structure represented by a formula:

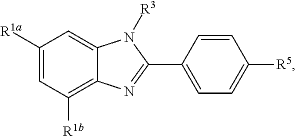

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein R³ is selected from hydrogen and C1-C4 alkyl; and wherein R⁵ is selected from halogen, —OH, and —CF₃, or a pharmaceutically acceptable salt thereof.

Also disclosed are methods of treating cancer in a subject, the method comprising administering to the subject an effective amount of at least one compound having a structure represented by a formula:

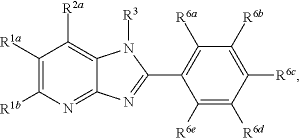

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein R^{2a} is selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; and wherein each of R^{6a}, R^{6b}, R^{6c}, R^{6d}, and R^{6e} is independently selected from hydrogen, halogen, —CF₃, and —OR²⁰, or a pharmaceutically acceptable salt thereof.

Also disclosed are pharmaceutical compositions comprising at least one of the above identified compounds or derivatives, and a pharmaceutically acceptable carrier.

Also disclosed are methods of treating a patient having a disease caused by or associated with abnormal Wnt signaling activity, which comprises administering to the patient an effective treatment amount of at least one of the above identified compounds or derivatives.

Also disclosed are methods of treating a patient having a cancer disease, which comprises administering to the patient an effective treatment amount of at least one of the above identified compounds or derivatives.

Also disclosed are methods of treating a patient for a precancerous condition or cancer, which comprises administering to the patient an effective treatment amount of at least one of the above identified compounds or derivatives.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more disorders prior to the administering step. In various aspects, the one or more disorders is a cancer.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of precancerous conditions and cancer prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “treating” refers to relieving the disease, disorder, or condition, i.e., causing regression of the disease, disorder, and/or condition. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

The term “comprising” (and its grammatical variations) as used herein is used in the inclusive sense of “having” or “including” and not in the exclusive sense of “consisting of.”

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

“Pharmaceutically acceptable salts” refer to derivatives of the disclosed compounds wherein the parent compound is modified by making acid or base salts thereof. The compounds of this disclosure form acid addition salts with a wide variety of organic and inorganic acids and include the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric acid, and the like. Salts derived from organic acids, such as aliphatic mono- and dicarboxylic acids, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, caprate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

It is understood that the compounds of the present disclosure relate to all optical isomers and stereo-isomers at the various possible atoms of the molecule, unless specified otherwise. Compounds may be separated or prepared as their pure enantiomers or diasteriomers by crystallization, chromatography or synthesis.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²)ₐ-, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹-, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula S(O)₂A¹, where A′ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹”, “R²”, “R³”, “Rⁿ”, where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o}C(O)OR^{o}); —N(R^{o})N(R^{o})C(O)R^{o}; —N(R^{o})N(R^{o})C(O)R^{o}; —N(R^{o}N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o}₂; SiR^{o}₃; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(R†)S(O)₂R†; wherein each R† is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono or bicyclic ring having 0-4 heteroatoms independently selected from nitrogen, oxygen, or sulfur.

Suitable substituents on the aliphatic group of R† are independently halogen, R^{•}, -(halon^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6 membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

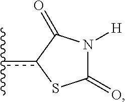

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkyl sulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. “Solvates” refers to the compound formed by the interaction of a solvent and a solute and includes hydrates. Solvates are usually crystalline solid adducts containing solvent molecules within the crystal structure, in either stoichiometric or nonstoichiometric proportions. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

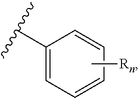

which is understood to be equivalent to a formula:

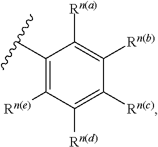

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further aspects, a structure of a compound can be represented by a formula:

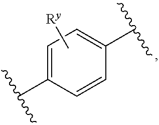

wherein R^{y} represents, for example, 0-2 independent substituents selected from A¹, A², and A³, which is understood to be equivalent to the groups of formulae:
wherein R^{y} represents 0 independent substituents

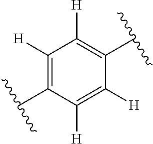

wherein R^{y} represents 1 independent substituent

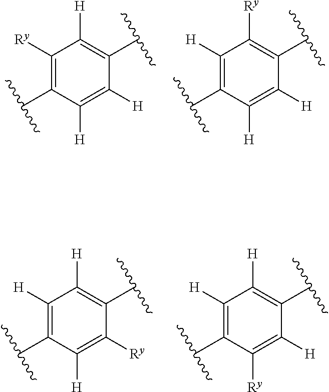

wherein R^{y} represents 2 independent substituents

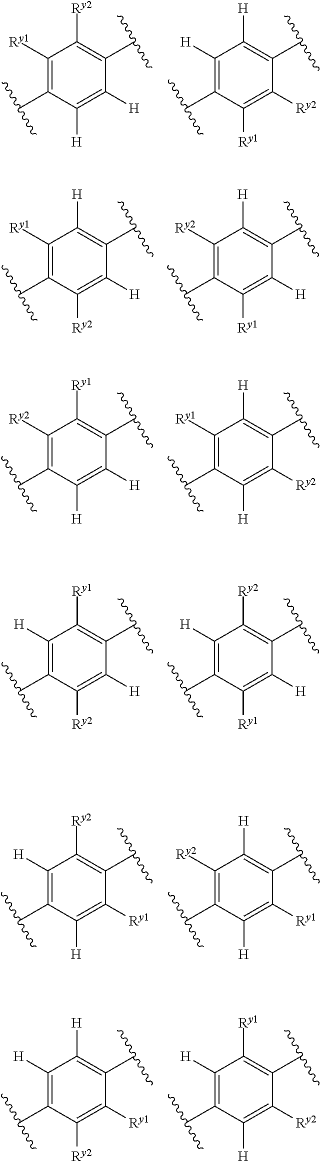

Again, by “independent substituents,” it is meant that each R substituent can be independently defined. For example, if in one instance R^{y1} is A¹, then R^{y2} is not necessarily A¹ in that instance.

In some further aspects, a structure of a compound can be represented by a formula,

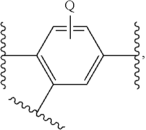

wherein, for example, Q comprises three substituents independently selected from hydrogen and A, which is understood to be equivalent to a formula:

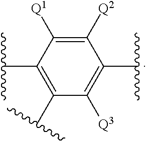

Again, by “independent substituents,” it is meant that each Q substituent is independently defined as hydrogen or A, which is understood to be equivalent to the groups of formulae:

wherein Q comprises three substituents independently selected from H and A

wherein Q comprises three substituents independently selected from H and A

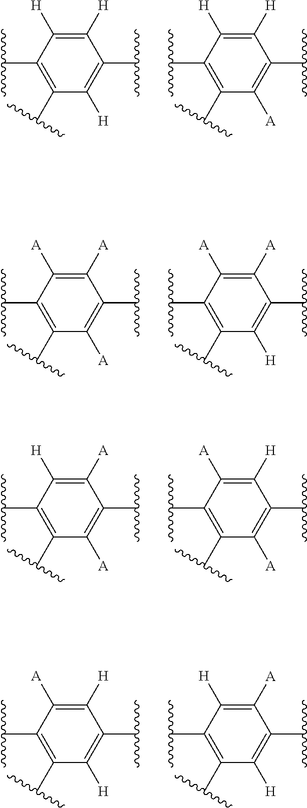

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating disorders associated with Wnt dysfunction, in particular cancers, such as pancreatic cancer, breast cancer, and colon cancer.

In one aspect, the disclosed compounds exhibit antagonism of Wnt.

In one aspect, the compounds of the invention are useful in inhibiting Wnt activity in a mammal. In a further aspect, the compounds of the invention are useful in inhibiting Wnt activity in at least one cell.

In one aspect, the compounds of the invention are useful in the treatment of disorders associated with Wnt dysfunction, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

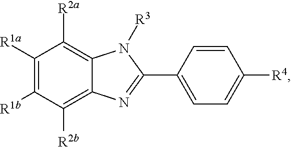

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

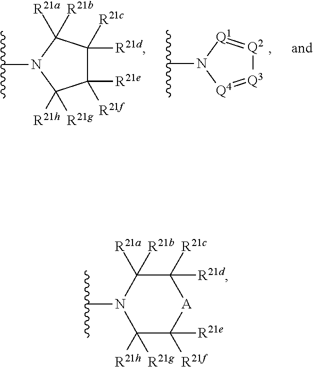

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; wherein each of Q¹, Q², Q³, and Q⁴, when present, is independently selected from CR³⁰ and N; and wherein each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are compounds having a structure represented by a formula:

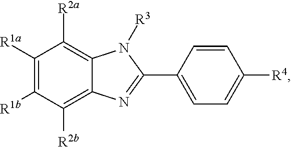

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

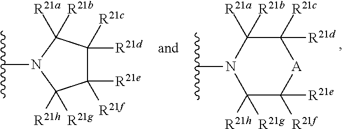

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; and wherein each of R^{21a}, R^{21b}, R^{21c}, R^{21d}, R^{21e}, R^{21f}, R^{21g}, and R^{21h} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are compounds having a structure selected from:

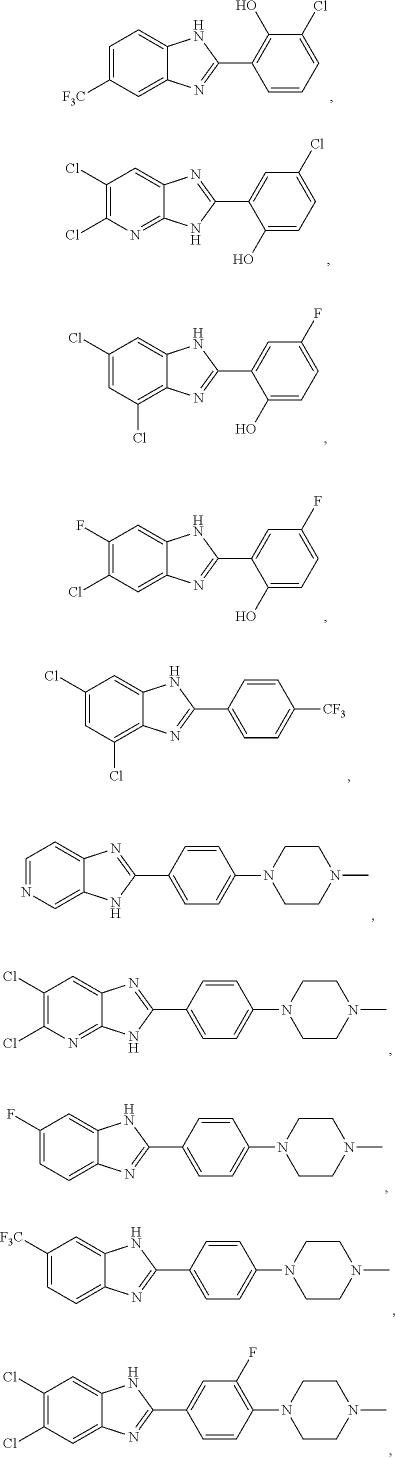

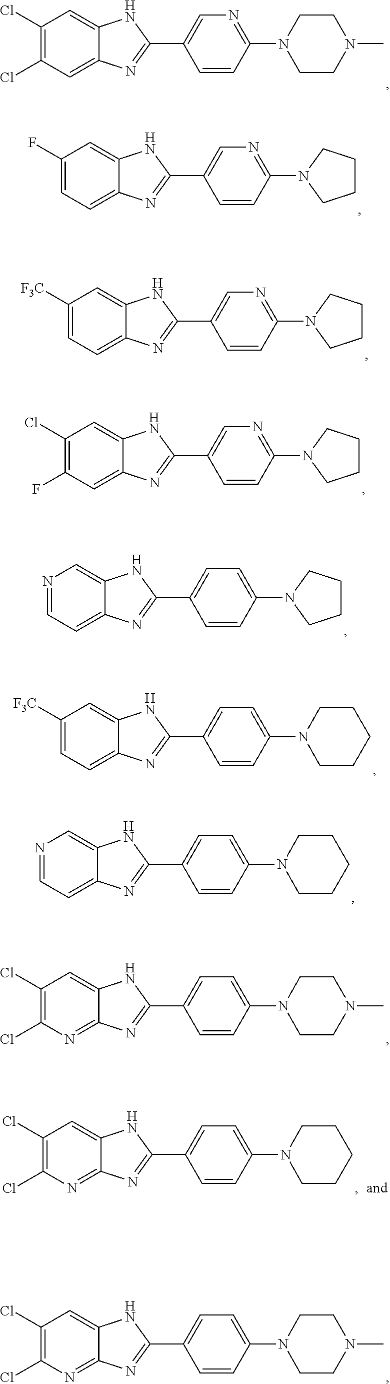

or a pharmaceutically acceptable salt thereof.

In a further aspect, the compound has a structure represented by a formula:

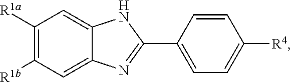

or a pharmaceutically acceptable salt thereof.

In a further aspect, the compound has a structure represented by a formula:

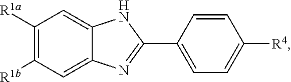

wherein R⁴ is a structure represented by a formula selected from:

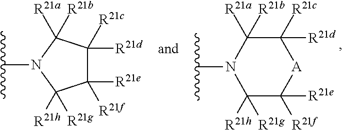

or a pharmaceutically acceptable salt thereof.

In a further aspect, the compound has a structure represented by a formula:

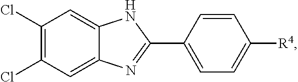

or a pharmaceutically acceptable salt thereof.

In a further aspect, the compound has a structure represented by a formula:

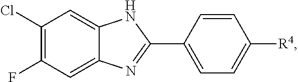

wherein R⁴ is a structure represented by a formula selected from:

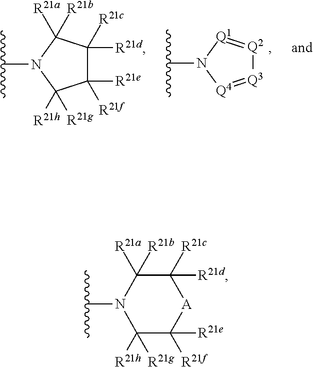

or a pharmaceutically acceptable salt thereof.

In a further aspect, the compound has a structure represented by a formula:

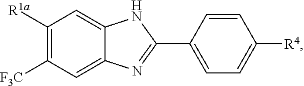

or a pharmaceutically acceptable salt thereof.

In a further aspect, the compound has a structure represented by a formula:

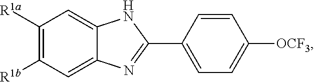

or a pharmaceutically acceptable salt thereof.

In a further aspect, the compound has a structure selected from:

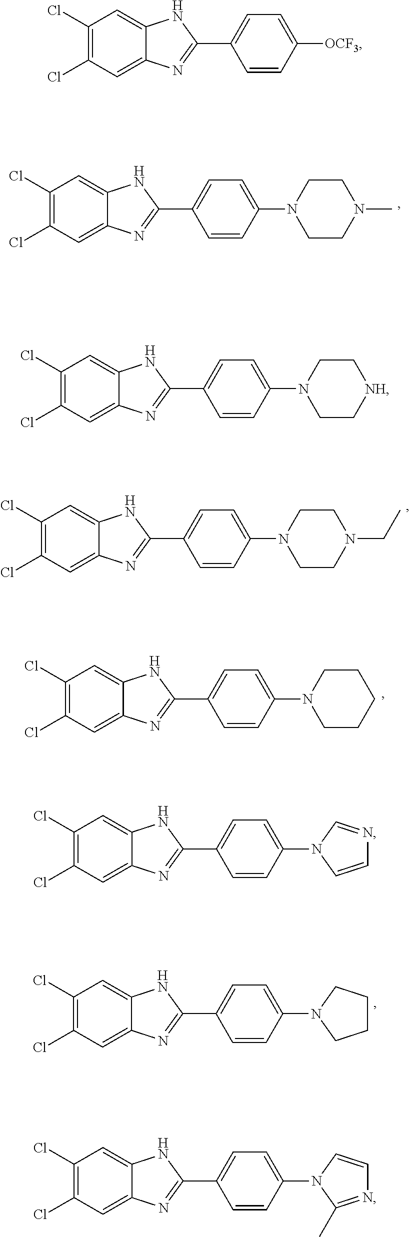

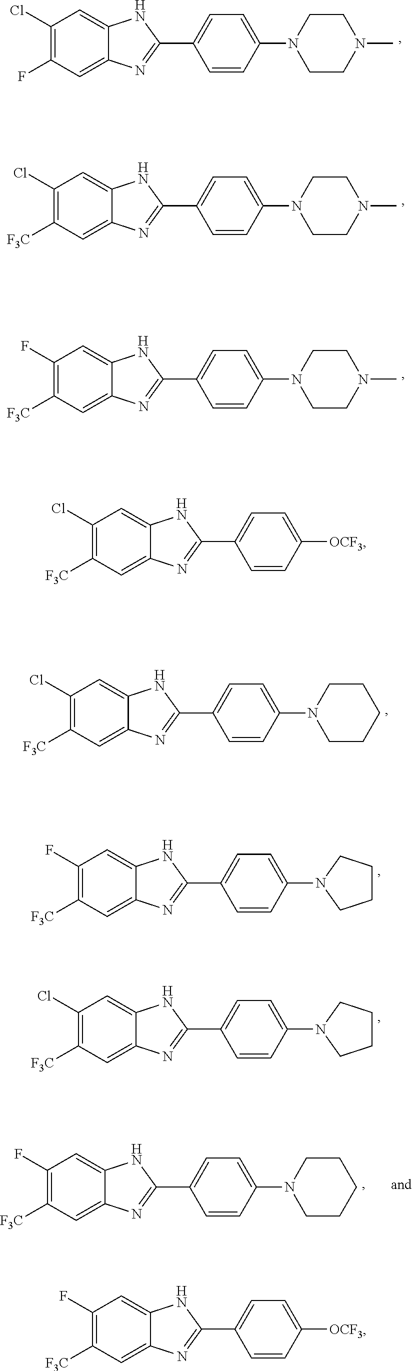

or a pharmaceutically acceptable salt thereof.

In a further aspect, the compound has a structure selected from:

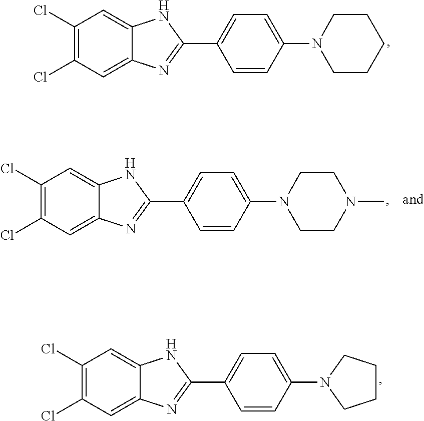 [SMILES: Clc1cc2nc(-c3ccc(N4CCCCC4)cc3)[nH]c2cc1Cl ; CN1CCN(c2ccc(-c3nc4cc(Cl)c(Cl)cc4[nH]3)cc2)CC1 ; Clc1cc2nc(-c3ccc(N4CCCC4)cc3)[nH]c2cc1Cl]

or a pharmaceutically acceptable salt thereof.

In a further aspect, the compound has a structure selected from:

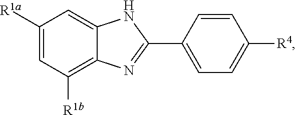

or a pharmaceutically acceptable salt thereof.

In a further aspect, the compound has a structure selected from:

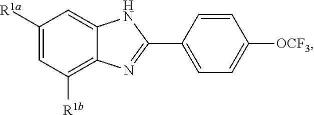

or a pharmaceutically acceptable salt thereof.

In a further aspect, the compound has a structure selected from:

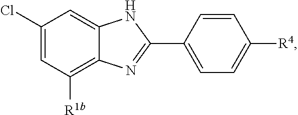

or a pharmaceutically acceptable salt thereof.

In a further aspect, the compound has a structure selected from:

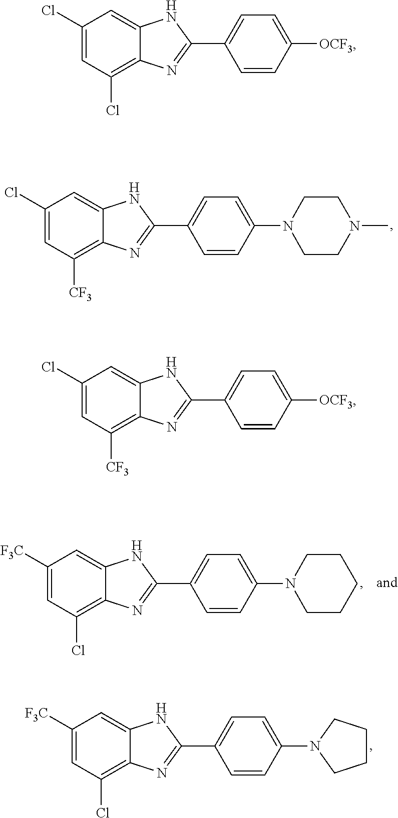 [SMILES: FC(F)(F)Oc1ccc(-c2nc3c(Cl)cc(Cl)cc3[nH]2)cc1 ; CN1CCN(c2ccc(-c3nc4c(C(F)(F)F)cc(Cl)cc4[nH]3)cc2)CC1 ; FC(F)(F)Oc1ccc(-c2nc3c(C(F)(F)F)cc(Cl)cc3[nH]2)cc1 ; FC(F)(F)c1cc(Cl)c2nc(-c3ccc(N4CCCCC4)cc3)[nH]c2c1 ; FC(F)(F)c1cc(Cl)c2nc(-c3ccc(N4CCCC4)cc3)[nH]c2c1]

or a pharmaceutically acceptable salt thereof.

a. A Groups

In one aspect, A, when present, is selected from O, NR³⁰, and CR^{31a}R^{31b}. In a further aspect, A, when present, is selected from O and NR³⁰. In a still further aspect, A, when present, is selected from O and CR^{31a}R^{31b}. In yet a further aspect, A, when present, is selected from NR³⁰ and CR^{31a}R^{31b}. In an even further aspect, A, when present, is O. In a still further aspect, A, when present, is NR³⁰. In yet a further aspect, A, when present, is CR^{31a}R^{31b}.

In a further aspect, A, when present, is selected from O, NH, NCH₃, CH₂, CHCH₃, and C(CH₃)₂. In a still further aspect, A, when present, is selected from O, NH, NCH₃, CH₂, and CHCH₃. In yet a further aspect, A, when present, is selected from O, NH, and CH₂. In an even further aspect, A, when present is NH. In a still further aspect, A, when present is NCH₃. In yet a further aspect, A, when present, is CH₂. In an even further aspect, A, when present, is CHCH₃. In a still further aspect, A, when present, is C(CH₃)₂.

a. Q¹, Q², Q³, and Q⁴ Groups

In one aspect, each of Q¹, Q², Q³, and Q⁴, when present, is independently selected from CR³⁰ and N. In a further aspect, each of Q¹, Q², Q³, and Q⁴, when present, is CR³⁰. In a still further aspect, each of Q¹, Q², Q³, and Q⁴, when present, is N.

In a further aspect, one of Q¹, Q², Q³, and Q⁴, when present, is N. In a still further aspect, two of Q¹, Q², Q³, and Q⁴, when present, is N. In yet a further aspect, three of Q¹, Q², Q³, and Q⁴, when present, is N. In an even further aspect, four of Q¹, Q², Q³, and Q⁴, when present, is N. In a still further aspect, none of Q¹, Q², Q³, and Q⁴, when present, is N.

b. R^{1A} and R^{1B} Groups

In one aspect, each of R^{1a} and R^{1b} is independently selected from halogen, —CF₃, and —OR²⁰. In a further aspect, each of R^{1a} and R^{1b} is independently selected from halogen and —CF₃. In a still further aspect, each of R^{1a} and R^{1b} is independently selected from halogen and —OR²⁰. In yet a further aspect, each of R^{1a} and R^{1b} is independently selected from —CF₃ and —OR²⁰. In an even further aspect, each of R^{1a} and R^{1b} is independently halogen. In a still further aspect, each of R^{1a} and R^{1b} is independently —OR²⁰. In yet a further aspect, each of R^{1a} and R^{1b} is —CF₃.

In a further aspect, each of R^{1a} and R^{1b} is independently selected from —Cl, —F, —CF₃, —OH, —OCH₃, and —OCF₃. In a still further aspect, each of R^{1a} and R^{1b} is independently selected from —Cl, —F, —CF₃, and —OCF₃.

In a further aspect, each of R^{1a} and R^{1b} is independently halogen. In a still further aspect, each of R^{1a} and R^{1b} is independently selected from —Cl and —F. In yet a further aspect, each of R^{1a} and R^{1b} is —Cl. In an even further aspect, each of R^{1a} and R^{1b} is F.

c. R^{2A} and R^{2B} Groups

In one aspect, each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy. In a further aspect, each of R^{2a} and R^{2b} is hydrogen.

In a further aspect, each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 monohaloalkoxy, and C1-C4 polyhaloalkoxy. In a still further aspect, each of R^{2a} and R^{2b} is independently selected from hydrogen, —Cl, —F, —CN, —OH, —SH, —NH₂, methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —OCH₂F, —OCH₂Cl, —OCH₂Br, —OCH₂CH₂F, —OCH₂CH₂Cl, —OCH₂CH₂Br, —O(CH₂)₂CH₂F, —O(CH₂)₂CH₂Cl, —O(CH₂)₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, —OCH₂CBr₃, —O(CH₂)₂CHF₂, —O(CH₂)₂CF₃, —O(CH₂)₂CHCl₂, —O(CH₂)₂CCl₃, —O(CH₂)₂CHBr₂, and —O(CH₂)₂CBr₃. In yet a further aspect, each of R^{2a} and R^{2b} is independently selected from hydrogen, —Cl, —F, —CN, —OH, —SH, —NH₂, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —OCH₂F, —OCH₂Cl, —OCH₂Br, —OCH₂CH₂F, —OCH₂CH₂Cl, —OCH₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, and —OCH₂CBr₃. In an even further aspect, each of R^{2a} and R^{2b} is independently selected from hydrogen, —Cl, —F, —CN, —OH, —SH, —NH₂, methyl, —CH₂F, —CH₂Cl, —CH₂Br, —CHF₂, —CF₃, —CHCl₂, —CCl₃, —CHBr₂, —CBr₃, —OCH₂F, —OCH₂Cl, —OCH₂Br, —OCHF₂, —OCF₃, —OCHCl₂, —OCCl₃, —OCHBr₂, and —OCBr₃.

In a further aspect, each of R^{2a} and R^{2b} is independently selected from hydrogen, halogen, —CN, —OH, —SH, —NH₂, C1-C4 alkyl, C1-C4 alkoxy, and C1-C4 thioalkoxy. In a still further aspect, each of R^{2a} and R^{2b} is independently selected from hydrogen, —Cl, —F, —CN, —OH, —SH, —NH₂, methyl, ethyl, n-propyl, i-propyl, —OCH₃, —OCH₂CH₃, —O(CH₂)₂CH₃, —SCH₃, —SCH₂CH₃, and —S(CH₂)₂CH₃. In yet a further aspect, each of R^{2a} and R^{2b} is independently selected from hydrogen, —Cl, —F, —CN, —OH, —SH, —NH₂, methyl, ethyl, —OCH₃, —OCH₂CH₃, —SCH₃, and —SCH₂CH₃. In an even further aspect, each of R^{2a} and R^{2b} is independently selected from hydrogen, —Cl, —F, —CN, —OH, —SH, —NH₂, methyl, —OCH₃, and —SCH₃.

In a further aspect, each of R^{2a} and R^{2b} is independently selected from hydrogen, halogen, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy. In a still further aspect, each of R^{2a} and R^{2b} is independently selected from hydrogen, —Cl, —F, methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂, —CHF₂, —CH₂, —CF₃, —CH₂ CHCl₂, —CH₂CCl₃, —CH₂CHBr₂, —CH₂CBr₃, —(CH₂)₂ CHF₂, —(CH₂)₂CF₃, —(CH₂)₂CHCl₂, —(CH₂)₂CCl₃, —(CH₂)₂CHBr₂, —(CH₂)₂CBr₃, —OCH₃, —OCH₂CH₃, —O(CH₂)₂CH₃, —SCH₃, —SCH₂CH₃, —S(CH₂)₂CH₃, —OCH₂F, —OCH₂Cl, —OCH₂Br, —OCH₂CH₂F, —OCH₂CH₂Cl, —OCH₂CH₂Br, —O(CH₂)₂CH₂F, —O(CH₂)₂CH₂Cl, —O(CH₂)₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, —OCH₂CBr₃, —O(CH₂)₂CHF₂, —O(CH₂)₂CF₃, —O(CH₂)₂CHCl₂, —O(CH₂)₂CCl₃, —O(CH₂)₂CHBr₂, and —O(CH₂)₂CBr₃. In yet a further aspect, each of R^{2a} and R^{2b} is independently selected from hydrogen, —Cl, —F, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂ CHCl₂, —CH₂CCl₃, —CH₂CHBr₂, —CH₂CBr₃, —OCH₃, —OCH₂CH₃, —SCH₃, —SCH₂CH₃, —OCH₂F, —OCH₂Cl, —OCH₂Br, —OCH₂CH₂F, —OCH₂CH₂Cl, —OCH₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, and —OCH₂CBr₃. In an even further aspect, each of R^{2a} and R^{2b} is independently selected from hydrogen, —Cl, —F, methyl, —CH₂F, —CH₂Cl, —CH₂Br, —CHF₂, —CF₃, —CHCl₂, —CCl₃, —CHBr₂, —CBr₃, —OCH₃, —SCH₃, —OCH₂F, —OCH₂Cl, —OCH₂Br, —OCHF₂, —OCF₃, —OCHCl₂, —OCCl₃, —OCHBr₂, and —OCBr₃.

d. R³ Groups

In one aspect, R³ is selected from hydrogen and C1-C4 alkyl. In a further aspect, R³ is hydrogen.

In a further aspect, R³ is selected from hydrogen, methyl, ethyl, n-propyl, i-propyl, n-butyl, t-butyl, s-butyl, and i-butyl. In a still further aspect, R³ is selected from hydrogen, methyl, ethyl, n-propyl, and i-propyl. In yet a further aspect, R³ is selected from hydrogen, methyl, and ethyl. In an even further aspect, R³ is selected from hydrogen and ethyl. In a still further aspect, R³ is selected from hydrogen and methyl.

In a further aspect, R³ is C1-C4 alkyl. In a still further aspect, R³ is selected from methyl, ethyl, n-propyl, and i-propyl. In yet a further aspect, R³ is selected from methyl, and ethyl. In an even further aspect, R³ is ethyl. In a still further aspect, R³ is methyl.

e. R⁴ Groups

In one aspect, R⁴ is selected from —OCF₃ and a structure represented by a formula selected from:

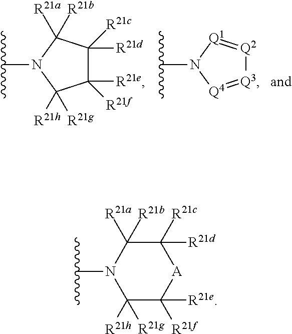

In a further aspect, R⁴ is —OCF₃.

In one aspect, R⁴ is selected from —OCF₃ and a structure represented by a formula selected from:

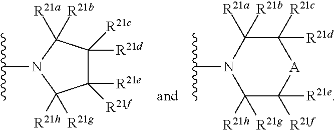

In a further aspect, R⁴ is a structure represented by a formula selected from:

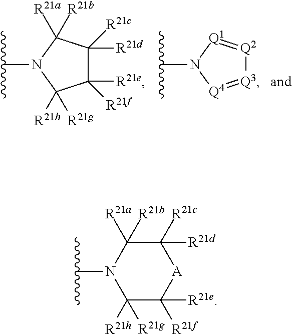

In a further aspect, R⁴ is a structure represented by a formula:

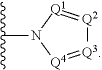

In a further aspect, R⁴ is a structure selected from:

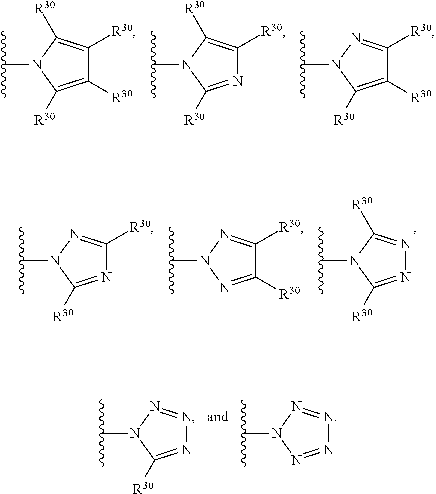

In a further aspect, R⁴ is a structure selected from:

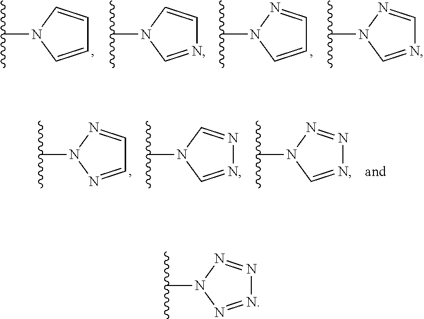

In a further aspect, R⁴ is a structure selected from:

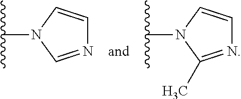

In a further aspect, R⁴ is a structure represented by a formula selected from:

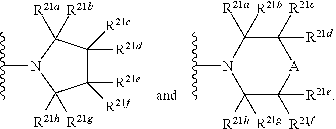

In a further aspect, R⁴ is a structure represented by a formula:

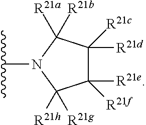

In a further aspect, R⁴ is a structure represented by a formula:

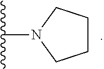 [SMILES: *N1CCCC1]

In a further aspect, R⁴ is a structure represented by a formula:

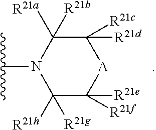

In a further aspect, R⁴ is a structure represented by a formula selected from:

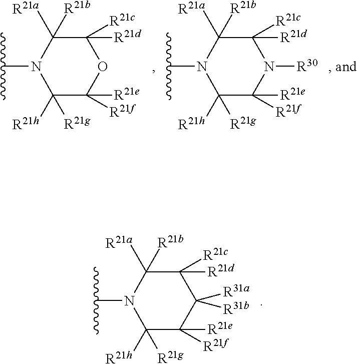

In a further aspect, R⁴ is a structure represented by a formula selected from:

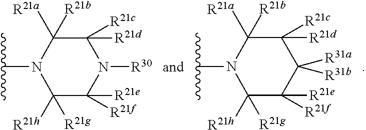

In a further aspect, R⁴ is a structure represented by a formula:

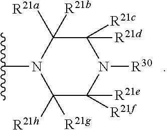

In a further aspect, R⁴ is a structure represented by a formula selected from:

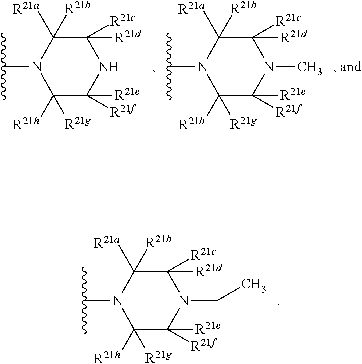

In a further aspect, R⁴ is a structure represented by a formula selected from:

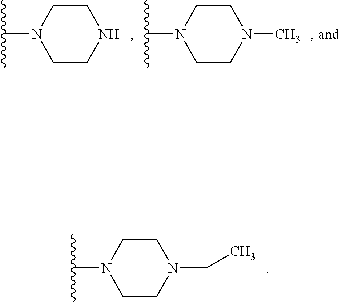

In a further aspect, R⁴ is a structure represented by a formula:

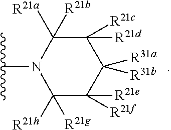

In a further aspect, R⁴ is a structure represented by a formula:

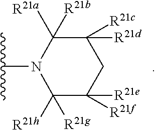

In a further aspect, R⁴ is a structure represented by a formula:

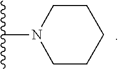 [SMILES: *N1CCCCC1]

f. R⁵ Groups

In one aspect, R⁵ is selected from halogen, —OH, and —CF₃. In a further aspect, R⁵ is selected from halogen and —CF₃. In a still further aspect, R⁵ is selected from halogen and —OH. In yet a further aspect, R⁵ is selected from —CF₃ and —OH. In an even further aspect, R⁵ is halogen. In a still further aspect, R⁵ is —OH. In yet a further aspect, R⁵ is —CF₃.

In a further aspect, R⁵ is selected from —Cl, —F, —CF₃, and —OH. In a still further aspect, R⁵ is selected from —Cl, —CF₃, and —OCF₃.

In a further aspect, R⁵ is halogen. In a still further aspect, R⁵ is selected from —Cl and —F. In yet a further aspect, R⁵ is —Cl. In an even further aspect, R⁵ is —F.

g. R^{6a}, R^{6B}, R^{6C}, R^{6D}, and R^{6E} Groups

In one aspect, each of R^{6a}, R^{6b}, R^{6c}, R^{6d}, and R^{6e} is independently selected from hydrogen, halogen, —CF₃, and —OR²⁰. In a further aspect, each of R^{6a}, R^{6b}, R^{6c}, R^{6d}, and R^{6e} is independently selected from hydrogen, halogen, —CF₃, and —OR²⁰, provided that at least one of R^{6a}, R^{6b}, R^{6c}, R^{6d}, and R^{6e} is not hydrogen. In a still further aspect, each of R^{6a}, R^{6b}, R^{6c}, R^{6d}, and R^{6e} is hydrogen.

In a further aspect, each of R^{6a}, R^{6b}, R^{6c}, R^{6d}, and R^{6e} is independently selected from hydrogen, —Cl, —F, —CF₃, —OH, —OCH₃, and —OCF₃. In a still further aspect, each of R^{6a}, R^{6b}, R^{6c}, R^{6d}, and R^{6e} is independently selected from hydrogen, —Cl, —F, —CF₃, and —OCF₃. In yet a further aspect, each of R^{6a}, R^{6b}, R^{6c}, R^{6d}, and R^{6e} is independently selected from hydrogen, —Cl, —F, and —CF₃. In an even further aspect, each of R^{6a}, R^{6b}, R^{6c}, R^{6d}, and R^{6e} is independently selected from hydrogen, —Cl, and —F. In a still further aspect, each of R^{6a}, R^{6b}, R^{6c}, R^{6d}, and R^{6e} is independently selected from hydrogen and —F. In yet a further aspect, each of R^{6a}, R^{6b}, R^{6c}, R^{6d}, and R^{6e} is independently selected from hydrogen and —Cl.

In a further aspect, each of R^{6a}, R^{6b}, R^{6d}, and R^{6e} is hydrogen and R^{6c} is selected from halogen, —CF₃, and —OR²⁰. In a still further aspect, each of R^{6a}, R^{6b}, R^{6d}, and R^{6e} is hydrogen and R^{6c} is selected from —Cl, —F, —CF₃, —OH, —OCH₃, and —OCF₃. In yet a further aspect, each of R^{6a}, R^{6b}, R^{6d}, and R^{6e} is hydrogen and R^{6c} is selected from —Cl, —F, —CF₃, and —OCF₃. In an even further aspect, each of R^{6a}, R^{6b}, R^{6d}, and R^{6e} is hydrogen and R^{6c} is selected from —Cl, —F, and —OCF₃. In a still further aspect, each of R^{6a}, R^{6b}, R^{6d}, and R^{6e} is hydrogen and R^{6c} is selected from —Cl and —F. In yet a further aspect, each of R^{6a}, R^{6b}, R^{6d}, and R^{6e} is hydrogen and R^{6c} is —OCF₃. In an even further aspect, each of R^{6a}, R^{6b}, R^{6d}, and R^{6e} is hydrogen and R^{6c} is —Cl. In a still further aspect, each of R^{6a}, R^{6b}, R^{6d}, and R^{6e} is hydrogen and R^{6c} is —F. In yet a further aspect, each of R^{6a}, R^{6b}, R^{6d}, and R^{6e} is hydrogen and R^{6c} is —CF₃.

h. R²⁰ Groups

In one aspect, each occurrence of R²⁰, when present, is independently selected from hydrogen, C1-C4 alkyl, C1-C4 monohaloalkyl, and C1-C4 polyhaloalkyl. In a further aspect, each occurrence of R²⁰, when present, is hydrogen.

In a further aspect, each occurrence of R²⁰, when present, is independently selected from hydrogen, methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, and —(CH₂)₂CBr₃. In a still further aspect, each occurrence of R²⁰, when present, is independently selected from hydrogen, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, and —CH₂CBr₃. In yet a further aspect, each occurrence of R²⁰, when present, is independently selected from hydrogen, methyl, —CH₂F, —CH₂Cl, —CH₂Br, —CHF₂, —CF₃, —CHCl₂, —CCl₃, —CHBr₂, —CBr₃.

In a further aspect, each occurrence of R²⁰, when present, is independently selected from hydrogen, C1-C4 monohaloalkyl, and C1-C4 polyhaloalkyl. In a still further aspect, each occurrence of R²⁰, when present, is independently selected from hydrogen, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, and —(CH₂)₂CBr₃. In yet a further aspect, each occurrence of R²⁰, when present, is independently selected from hydrogen, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, and —CH₂CBr₃. In an even further aspect, each occurrence of R²⁰, when present, is independently selected from hydrogen, —CH₂F, —CH₂Cl, —CH₂Br, —CHF₂, —CF₃, —CHCl₂, —CCl₃, —CHBr₂, —CBr₃. In a still further aspect, each occurrence of R²⁰, when present, is independently selected from hydrogen, —CF₃, —CCl₃, and —CBr₃. In yet a further aspect, each occurrence of R²⁰, when present, is independently selected from hydrogen and —CF₃.

In a further aspect, each occurrence of R²⁰, when present, is independently selected from hydrogen and C1-C4 alkyl. In a still further aspect, each occurrence of R²⁰, when present, is independently selected from hydrogen, methyl, ethyl, n-propyl, and i-propyl. In yet a further aspect, each occurrence of R²⁰, when present, is independently selected from hydrogen, methyl, and ethyl. In an even further aspect, each occurrence of R²⁰, when present, is independently selected from hydrogen and ethyl. In a still further aspect, each occurrence of R²⁰, when present, is independently selected from hydrogen and methyl.

i. R^{21a}, R^{21b}, R^{21c}, R^{21d}, R^{21e}, R^{21f}, R^{21g}, and R^{21h} Groups

In one aspect, each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy. In a further aspect, each of R^{21a}, R^{21b}, R^{21c}, R^{21d}, R^{21e}, R^{21f}, R^{21g}, and R^{21h}, when present, is hydrogen.

In a further aspect, each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 monohaloalkoxy, and C1-C4 polyhaloalkoxy. In a still further aspect, each of lea, R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, ethyl, n-propyl, i-propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, (CH₂)₂CF₃, —(CH₂)₂CHCl₂, —(CH₂)₂CCl₃, —(CH₂)₂CHBr₂, —(CH₂)₂CBr₃, —OCH₂F, —OCH₂Cl, —OCH₂Br, —OCH₂CH₂F, —OCH₂CH₂Cl, —OCH₂CH₂Br, —O(CH₂)₂CH₂F, —O(CH₂)₂CH₂Cl, —O(CH₂)₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, —OCH₂CBr₃, —O(CH₂)₂CHF₂, —O(CH₂)₂CF₃, —O(CH₂)₂CHCl₂, —O(CH₂)₂CCl₃, —O(CH₂)₂CHBr₂, and —O(CH₂)₂CBr₃. In yet a further aspect, each of R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —OCH₂F, —OCH₂Cl, —OCH₂Br, —OCH₂CH₂F, —OCH₂CH₂Cl, —OCH₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, and —OCH₂CBr₃. In an even further aspect, each of R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, —CH₂F, —CH₂Cl, —CH₂Br, —CHF₂, —CF₃, —CHCl₂, —CCl₃, —CHBr₂, —CBr₃, —OCH₂F, —OCH₂Cl, —OCH₂Br, —OCHF₂, —OCF₃, —OCHCl₂, —OCCl₃, —OCHBr₂, and —OCBr₃.

In a further aspect, each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 alkoxy, and C1-C4 thioalkoxy. In a still further aspect, each of R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, ethyl, n-propyl, i-propyl, —OCH₃, —OCH₂CH₃, —O(CH₂)₂CH₃, —SCH₃, —SCH₂CH₃, and —S(CH₂)₂CH₃. In yet a further aspect, each of R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, ethyl, —OCH₃, —OCH₂CH₃, —SCH₃, and —SCH₂CH₃. In an even further aspect, each of R^{21a}, R^{21b}, R^{21c}, R^{21d}, R^{21e}, R^{21f}, R^{21g}, and R^{21h}, when present, is independently selected from hydrogen, —Cl, —F, —CN, —OH, —SH, —NH₂, methyl, —OCH₃, and —SCH₃.

In a further aspect, each of R^{21a}, R^{21b}, R^{21c}, R^{21d}, R^{21e}, R^{21f}, R^{21g}, and R^{21h}, when present, is independently selected from hydrogen, halogen, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy. In a still further aspect, each of R^{21a}, R^{21b}, R^{21c}, R^{21d}, R^{21e}, R^{21f}, R^{21g}, and R^{21h}, when present, is independently selected from hydrogen, —Cl, —F, methyl, ethyl, n-propyl, propyl, —CH₂F, —CH₂Cl, —CH₂Br, —CH₂CH₂F, —CH₂CH₂Cl, —CH₂CH₂Br, —(CH₂)₂CH₂F, —(CH₂)₂CH₂Cl, —(CH₂)₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —(CH₂)₂CHF₂, —(CH₂)₂CF₃, —(CH₂)₂CHCl₂, —(CH₂)₂CCl₃, —(CH₂)₂CHBr₂, —(CH₂)₂CBr₃, —OCH₃, —OCH₂CH₃, —O(CH₂)₂CH₃, —SCH₃, —SCH₂CH₃, —S(CH₂)₂CH₃, —OCH₂F, —OCH₂Cl, —OCH₂Br, —OCH₂CH₂F, —OCH₂CH₂Cl, —OCH₂CH₂Br, —O(CH₂)₂CH₂F, —O(CH₂)₂CH₂Cl, —O(CH₂)₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, —OCH₂CBr₃, —O(CH₂)₂CHF₂, —O(CH₂)₂CF₃, —O(CH₂)₂CHCl₂, —O(CH₂)₂CCl₃, —O(CH₂)₂CHBr₂, and —O(CH₂)₂CBr₃. In yet a further aspect, each of R^{21a}, R^{21b}, R^{21c}, R^{21d}, R^{21e}, R^{21f}, R^{21g}, and R^{21h}, when present, is independently selected from hydrogen, —Cl, —F, methyl, ethyl, —CH₂F, —CH₂Cl, —CH₂Br, —CH₂CH₂F, —CH₂CH₂Cl, —CH₂CH₂Br, —CHF₂, —CF₃, —CHCl₂, —CCl₃, —CHBr₂, —CBr₃, —CH₂CHF₂, —CH₂CF₃, —CH₂CHCl₂, —CH₂CCl₃, —CH₂CHBr₂, —CH₂CBr₃, —OCH₃, —OCH₂CH₃, —SCH₃, —SCH₂CH₃, —OCH₂F, —OCH₂Cl, —OCH₂Br, —OCH₂CH₂F, —OCH₂CH₂Cl, —OCH₂CH₂Br, —OCHF₂, —OCF₃, —OCHCl₂, —OCCl₃, —OCHBr₂, —OCBr₃, —OCH₂CHF₂, —OCH₂CF₃, —OCH₂CHCl₂, —OCH₂CCl₃, —OCH₂CHBr₂, and —OCH₂CBr₃. In an even further aspect, each of R^{21a}, R^{21b}, R^{21c}, R^{21d}, R^{21e}, R^{21f}, R^{21g}, and R^{21h}, when present, is independently selected from hydrogen, —Cl, —F, methyl, —CH₂F, —CH₂Cl, —CH₂Br, —CHF₂, —CF₃, —CHCl₂, —CCl₃, —CHBr₂, —CBr₃, —OCH₃, —SCH₃, —OCH₂F, —OCH₂Cl, —OCH₂Br, —OCHF₂, —OCF₃, —OCHCl₂, —OCCl₃, —OCHBr₂, and —OCBr₃.

j. R³⁰ Groups

In one aspect, R³⁰, when present, is selected from hydrogen and C1-C4 alkyl. In a further aspect, R³⁰, when present, is hydrogen.

In a further aspect, R³⁰, when present, is selected from hydrogen, methyl, ethyl, n-propyl, i-propyl, n-butyl, t-butyl, s-butyl, and i-butyl. In a still further aspect, R³⁰, when present, is selected from hydrogen, methyl, ethyl, n-propyl, and i-propyl. In yet a further aspect, R³⁰, when present, is selected from hydrogen, methyl, and ethyl. In an even further aspect, R³⁰, when present, is selected from hydrogen and ethyl. In a still further aspect, R³⁰, when present, is selected from hydrogen and methyl.

In a further aspect, R³⁰, when present, is C1-C4 alkyl. In a still further aspect, R³⁰, when present, is selected from methyl, ethyl, n-propyl, and i-propyl. In yet a further aspect, R³⁰, when present, is selected from methyl, and ethyl. In an even further aspect, R³⁰, when present, is ethyl. In a still further aspect, R³⁰, when present, is methyl.

k. R^{31A} and R^{31B} Groups

In one aspect, each of R^{31a} and R^{31b}, when present, is independently selected from hydrogen and C1-C4 alkyl. In a further aspect, each of R^{31a} and R^{31b}, when present, is hydrogen.

In a further aspect, each of R^{31a} and R^{31b}, when present, is independently selected from hydrogen, methyl, ethyl, n-propyl, i-propyl, n-butyl, t-butyl, s-butyl, and i-butyl. In a still further aspect, each of R^{31a} and R^{31b}, when present, is independently selected from hydrogen, methyl, ethyl, n-propyl, and i-propyl. In yet a further aspect, each of R^{31a} and R^{31b}, when present, is independently selected from hydrogen, methyl, and ethyl. In an even further aspect, each of R^{31a} and R^{31b}, when present, is independently selected from hydrogen and ethyl. In a still further aspect, each of R^{31a} and R^{31b}, when present, is independently selected from hydrogen and methyl.

In a further aspect, each of R^{31a} and R^{31b}, when present, is independently C1-C4 alkyl. In a still further aspect, each of R^{31a} and R^{31b}, when present, is independently selected from methyl, ethyl, n-propyl, and i-propyl. In yet a further aspect, each of R^{31a} and R^{31b}, when present, is independently selected from methyl, and ethyl. In an even further aspect, each of R^{31a} and R^{31b}, when present, is ethyl. In a still further aspect, each of R^{31a} and R^{31b}, when present, is methyl.

### 2. Example Compounds

In one aspect, a compound can be present as one or more of the following structures:

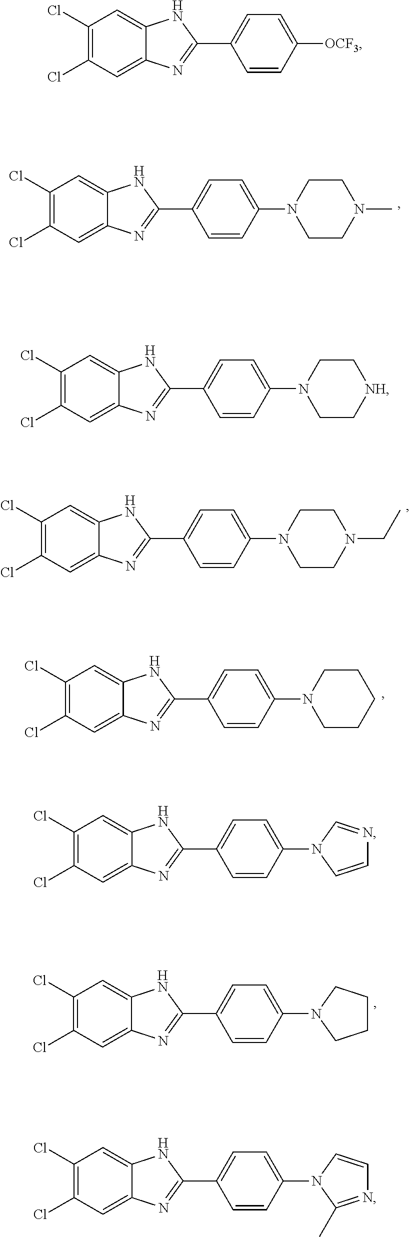

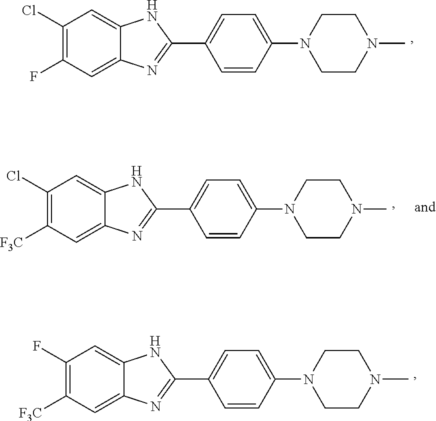 [SMILES: CN1CCN(c2ccc(-c3nc4cc(F)c(Cl)cc4[nH]3)cc2)CC1 ; CN1CCN(c2ccc(-c3nc4cc(C(F)(F)F)c(Cl)cc4[nH]3)cc2)CC1 ; CN1CCN(c2ccc(-c3nc4cc(C(F)(F)F)c(F)cc4[nH]3)cc2)CC1]

or a pharmaceutically acceptable salt thereof.

In one aspect, a compound can be present as the following structure:

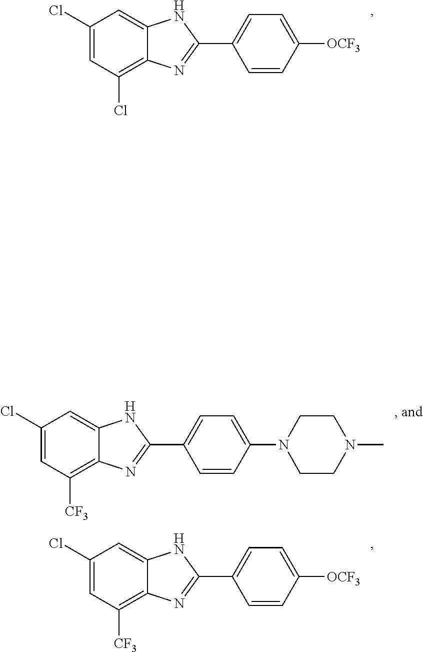

or a pharmaceutically acceptable salt thereof.

In one aspect, a compound can be present as one or more of the following structures:

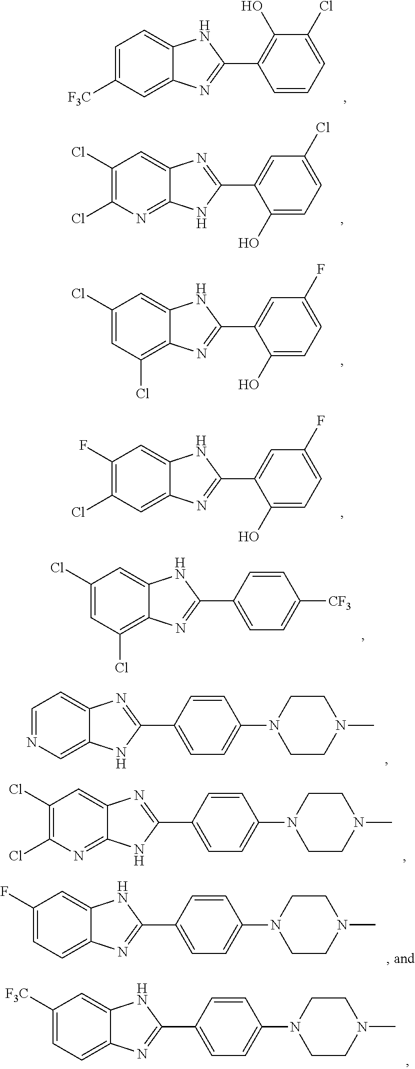

or a pharmaceutically acceptable salt thereof.

In one aspect, a compound cannot be present as a structure selected from:

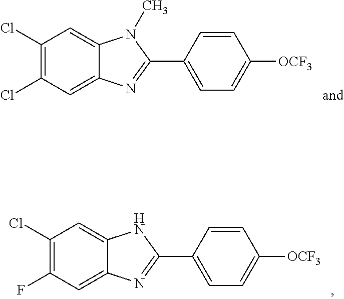 [SMILES: Cn1c(-c2ccc(OC(F)(F)F)cc2)nc2cc(Cl)c(Cl)cc21 ; Fc1cc2nc(-c3ccc(OC(F)(F)F)cc3)[nH]c2cc1Cl]

or a pharmaceutically acceptable salt thereof.

### 3. Prophetic Compound Examples

The following compound examples are prophetic, and can be prepared using the synthesis methods described herein above and other general methods as needed as would be known to one skilled in the art. It is anticipated that the prophetic compounds would be active as Wnt antagonists, and such activity can be determined using the assay methods described herein.

In one aspect, a compound can be selected from:

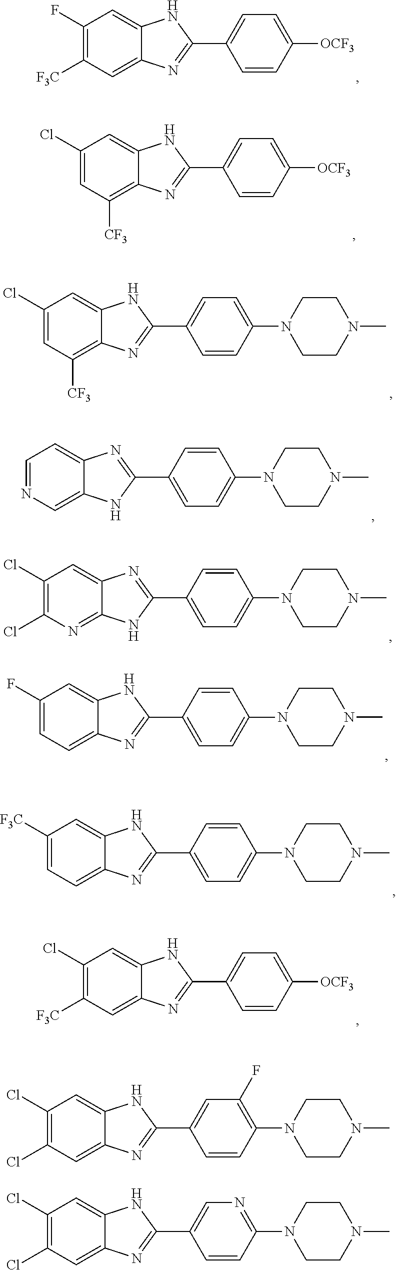

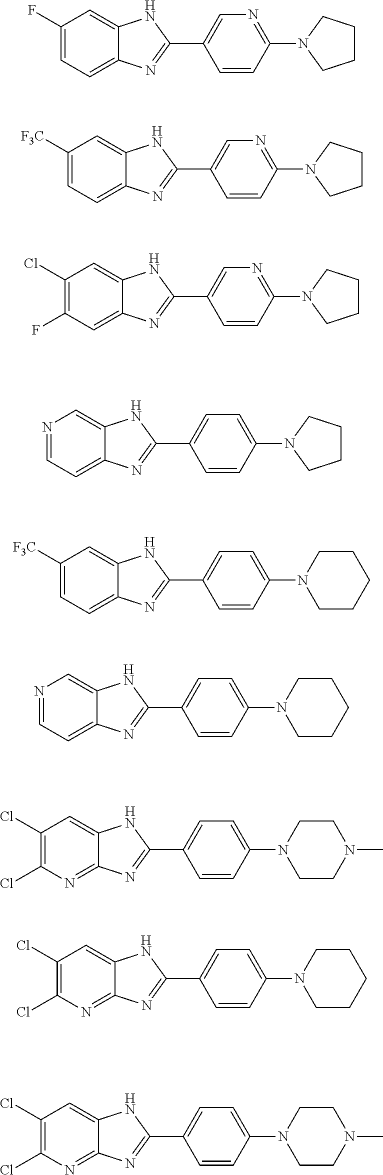

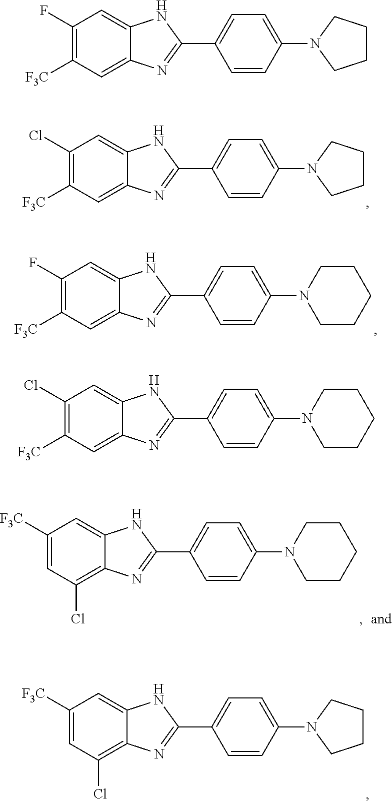 [SMILES: Fc1cc2[nH]c(-c3ccc(N4CCCC4)cc3)nc2cc1C(F)(F)F ; FC(F)(F)c1cc2nc(-c3ccc(N4CCCC4)cc3)[nH]c2cc1Cl ; Fc1cc2[nH]c(-c3ccc(N4CCCCC4)cc3)nc2cc1C(F)(F)F ; FC(F)(F)c1cc2nc(-c3ccc(N4CCCCC4)cc3)[nH]c2cc1Cl ; FC(F)(F)c1cc(Cl)c2nc(-c3ccc(N4CCCCC4)cc3)[nH]c2c1 ; FC(F)(F)c1cc(Cl)c2nc(-c3ccc(N4CCCC4)cc3)[nH]c2c1]

or a pharmaceutically acceptable salt thereof.

In one aspect, a compound can be selected from:

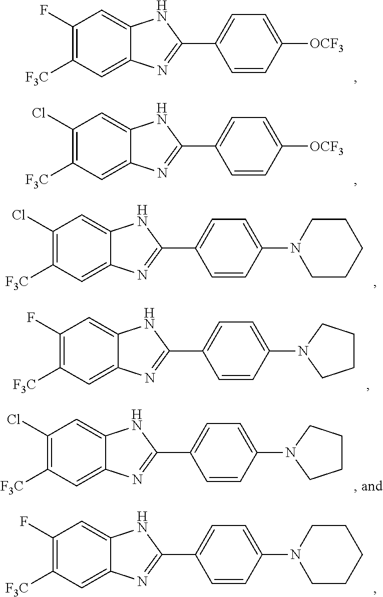

or a pharmaceutically acceptable salt thereof.

In one aspect, a compound can be selected from:

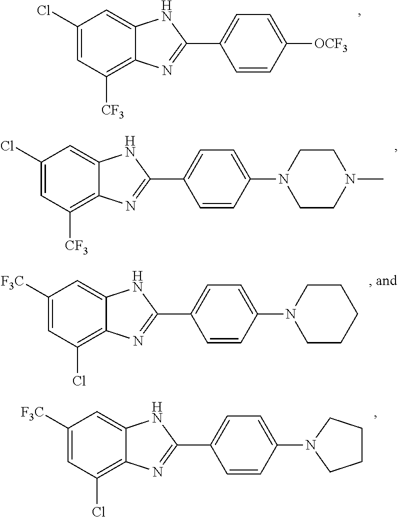

or a pharmaceutically acceptable salt thereof.

In one aspect, a compound can be selected from:

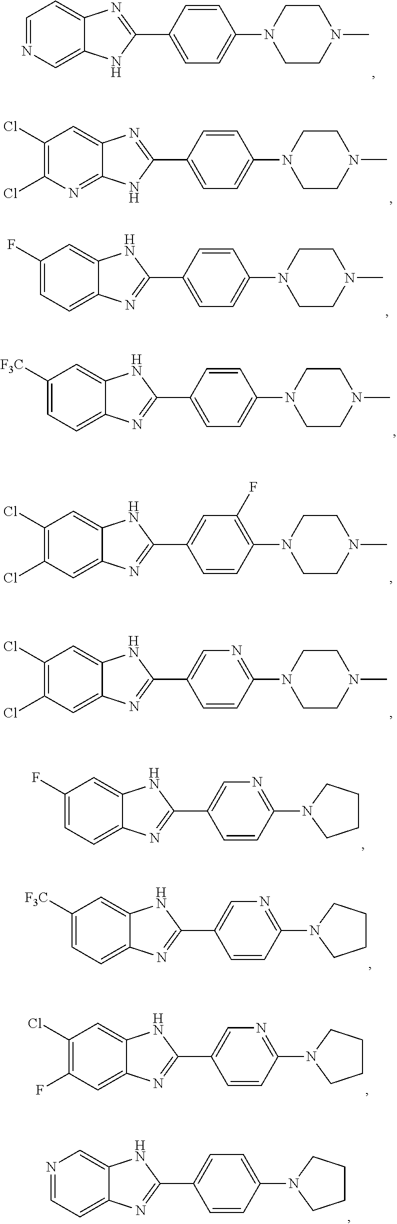

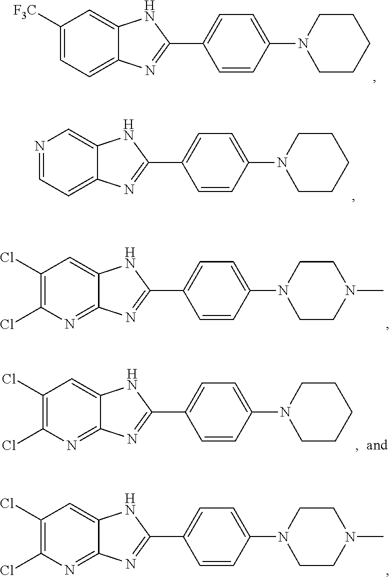 [SMILES: FC(F)(F)c1ccc2nc(-c3ccc(N4CCCCC4)cc3)[nH]c2c1 ; c1cc2nc(-c3ccc(N4CCCCC4)cc3)[nH]c2cn1 ; CN1CCN(c2ccc(-c3nc4nc(Cl)c(Cl)cc4[nH]3)cc2)CC1 ; Clc1cc2[nH]c(-c3ccc(N4CCCCC4)cc3)nc2nc1Cl ; CN1CCN(c2ccc(-c3nc4nc(Cl)c(Cl)cc4[nH]3)cc2)CC1]

or a pharmaceutically acceptable salt thereof.

### C. Pharmaceutical Compositions

In one aspect, the invention relates to pharmaceutical compositions comprising at least one disclosed compound and a pharmaceutically acceptable carrier. In a further aspect, a pharmaceutical composition can be provided comprising a therapeutically effective amount of at least one disclosed compound. In a still further aspect, a pharmaceutical composition can be provided comprising a prophylactically effective amount of at least one disclosed compound. In yet a further aspect, the invention relates to pharmaceutical compositions comprising a pharmaceutically acceptable carrier and a compound, wherein the compound is present in an effective amount.

The compounds are active against Wnt, and generally have IC₅₀ values against Wnt ranging from 0.0001 μM to 10 μM. IC₅₀ refers to the concentration of the compound that is required for 50% antagonism or inhibition of Wnt. IC₅₀ also refers to the concentration of a substance that is required for 50% antagonism or inhibition of Wnt in vivo. The activity of the compounds, including IC₅₀, is determined according to the procedures discussed below in the Examples section.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et. al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In a further aspect, the pharmaceutical composition is administered to a mammal. In a still further aspect, the mammal is a human. In an even further aspect, the human is a patient.

In a further aspect, the pharmaceutical composition is administered following identification of the mammal in need of treatment of a disorder associated with Wnt dysfunction. In a still further aspect, the mammal has been diagnosed with a need for treatment of a disorder associated with Wnt dysfunction prior to the administering step.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The benzimidazole compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcelluslose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyldialkylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the inhibition of Wnt signaling. The method also includes the administration of a therapeutically effect amount of the compound for the treatment of patient having a predisposition for being afflicted with cancer. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the cancer.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In a further aspect, the composition further comprises at least one agent known to treat a disorder associated with Wnt dysfunction. In a still further aspect, at least one agent known to treat a disorder associated with Wnt dysfunction, wherein the disorder is a cancer.

In a further aspect, the composition further comprises at least one agent known to have a side effect of increasing the risk of a disorder associated with Wnt dysfunction.

It is understood that the disclosed compositions can be prepared from the disclosed compounds. It is also understood that the disclosed compositions can be employed in the disclosed methods of using.

### D. Methods of Making the Compounds

In various aspects, the inventions relates to methods of making compounds useful to treat disorders associated with Wnt dysfunction. Thus, in one aspect, disclosed are methods of making a compound having a structure represented by a formula:

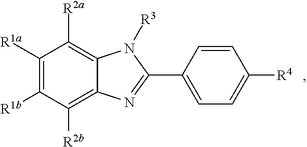

wherein each of R^{1a} and R^{1b} is independently selected from halogen, —CF₃, and —OR²⁰, wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

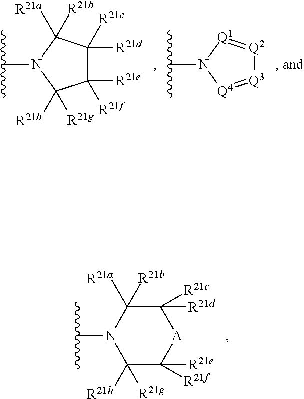

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; wherein each of Q¹, Q², Q³, and Q⁴, when present, is independently selected from CR³⁰ and N; and wherein each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are methods of making a compound having a structure represented by a formula:

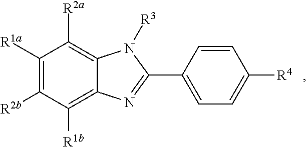

wherein each of R^{1a} and R^{1b} is independently selected from halogen, —CF₃, and —OR²⁰; wherein each occurrence of R²⁰, when present, is independently selected from hydrogen, C1-C4 alkyl, C1-C4 monohaloalkyl, and C1-C4 polyhaloalkyl; wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy; wherein R³ is selected from hydrogen and C1-C4 alkyl; wherein R⁴ is selected from —OCF₃ and a structure represented by a formula selected from:

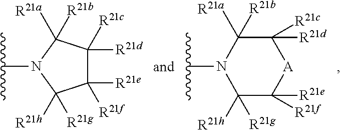

wherein A, when present, is selected from O, NR³⁰, and CR^{31a}R^{31b}; wherein R³⁰, when present, is selected from hydrogen and C1-C4 alkyl; wherein each of R^{31a} and R^{31b}, when present, is independently selected from hydrogen and C1-C4 alkyl; and wherein each of R^{21a}, R^{21b}, R^{21c}, R^{21d}, R^{21e}, R^{21f}, R^{21g}, and R^{21h} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy, or a pharmaceutically acceptable salt thereof.

Also disclosed are methods of making a compound having a structure selected from:

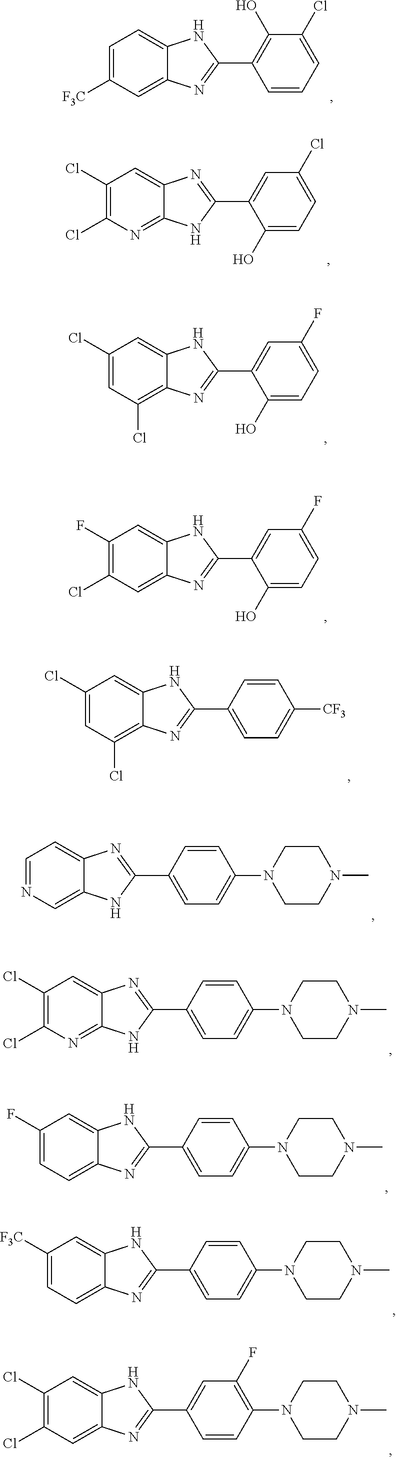

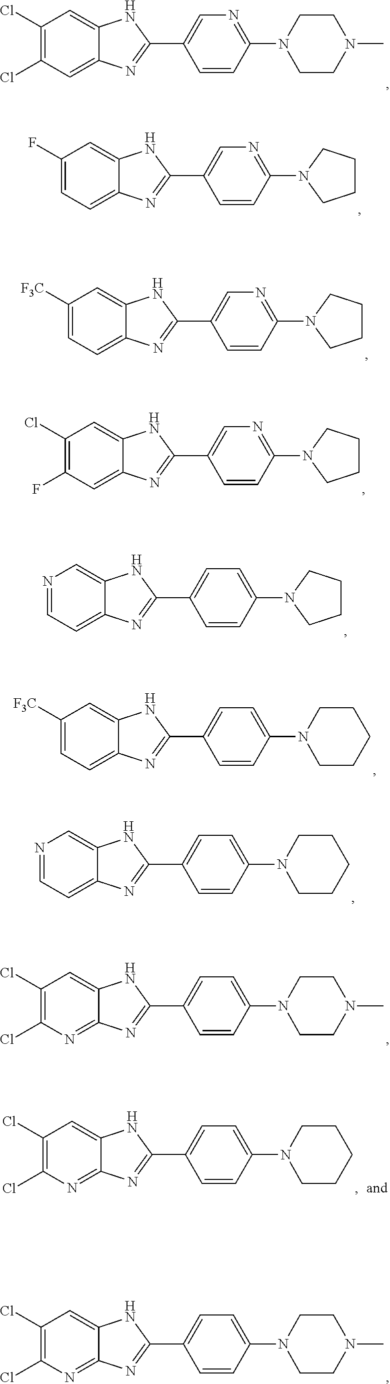

or a pharmaceutically acceptable salt thereof.

Compounds according to the present disclosure can, for example, be prepared by the several methods outlined below. A practitioner skilled in the art will understand the appropriate use of protecting groups [see: Greene and Wuts, Protective Groups in Organic Synthesis] and the preparation of known compounds found in the literature using the standard methods of organic synthesis. There may come from time to time the need to rearrange the order of the recommended synthetic steps, however this will be apparent to the judgment of a chemist skilled in the art of organic synthesis. The following examples are provided so that the invention might be more fully understood, are illustrative only, and should not be construed as limiting.

In one aspect, the disclosed compounds comprise the products of the synthetic methods described herein. In a further aspect, the disclosed compounds comprise a compound produced by a synthetic method described herein. In a still further aspect, the invention comprises a pharmaceutical composition comprising a therapeutically effective amount of the product of the disclosed methods and a pharmaceutically acceptable carrier. In a still further aspect, the invention comprises a method for manufacturing a medicament comprising combining at least one compound of any of disclosed compounds or at least one product of the disclosed methods with a pharmaceutically acceptable carrier or diluent.

### 1. Route I

In one aspect, benzimidazole analogs can be prepared as shown below.

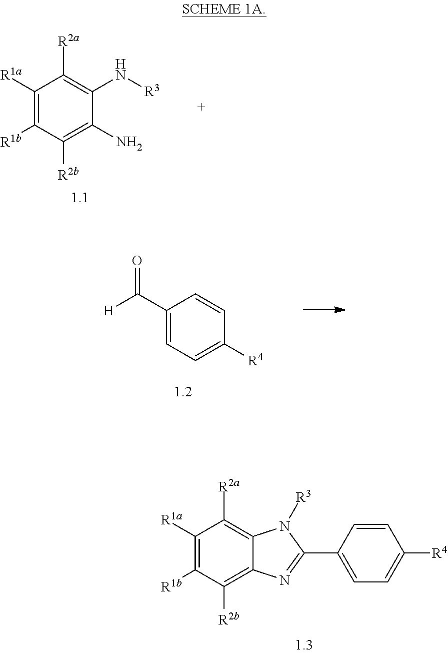

Compounds are represented in generic form, with substituents as noted in compound descriptions elsewhere herein. A more specific example is set forth below.

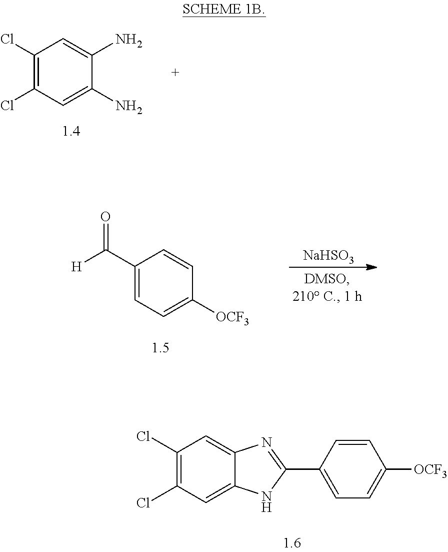 [SMILES: Nc1cc(Cl)c(Cl)cc1N ; O=Cc1ccc(OC(F)(F)F)cc1 ; "NaHSO3" ; FC(F)(F)Oc1ccc(-c2nc3cc(Cl)c(Cl)cc3[nH]2)cc1]

In one aspect, compounds of type 1.6, and similar compounds, can be prepared according to reaction Scheme 1B above. Thus, compounds of type 1.3 can be prepared by a condensation reaction of an appropriate diamine, e.g., 1.1 as shown above, and an appropriate benzaldehyde, e.g., 1.2 as shown above. Appropriate diamines and appropriate benzaldehydes are commercially available or prepared by methods known to one skilled in the art. The condensation reaction is carried out in the presence of an appropriate base, e.g., sodium bisulfite, in an appropriate solvent, e.g., dimethylsulfoxide, at an appropriate temperature, e.g., 210° C., for an appropriate period of time, e.g., 1 hour. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted benzimidazoles similar to Formula 1.6.

### 2. Route II

In one aspect, benzimidazole analogs can be prepared as shown below.

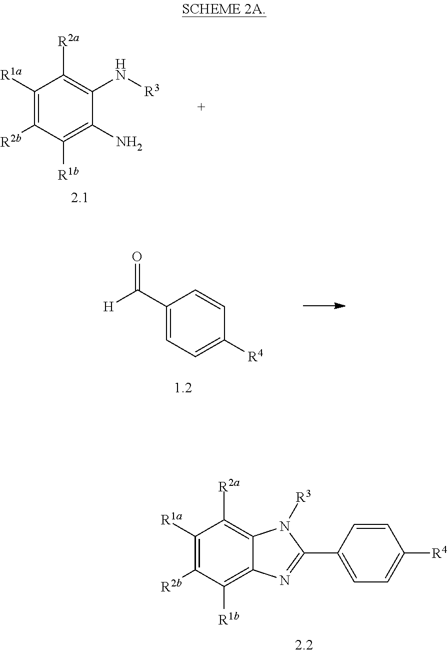

Compounds are represented in generic form, with substituents as noted in compound descriptions elsewhere herein. A more specific example is set forth below.

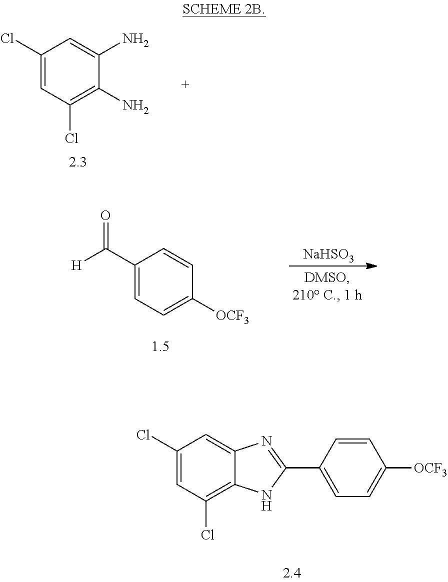 [SMILES: Nc1cc(Cl)cc(Cl)c1N ; O=Cc1ccc(OC(F)(F)F)cc1 ; "NaHSO3" ; FC(F)(F)Oc1ccc(-c2nc3cc(Cl)cc(Cl)c3[nH]2)cc1]

In one aspect, compounds of type 2.4, and similar compounds, can be prepared according to reaction Scheme 1B above. Thus, compounds of type 2.2 can be prepared by a condensation reaction of an appropriate diamine, e.g., 2.1 as shown above, and an appropriate benzaldehyde, e.g., 1.2 as shown above. Appropriate diamines and appropriate benzaldehydes are commercially available or prepared by methods known to one skilled in the art. The condensation reaction is carried out in the presence of an appropriate base, e.g., sodium bisulfite, in an appropriate solvent, e.g., dimethylsulfoxide, at an appropriate temperature, e.g., 210° C., for an appropriate period of time, e.g., 1 hour. As can be appreciated by one skilled in the art, the above reaction provides an example of a generalized approach wherein compounds similar in structure to the specific reactants above (compounds similar to compounds of type 2.1 and 1.2), can be substituted in the reaction to provide substituted benzimidazoles similar to Formula 2.4.

### 3. Route III

In one aspect, benzimidazole analogs can be prepared as shown below.

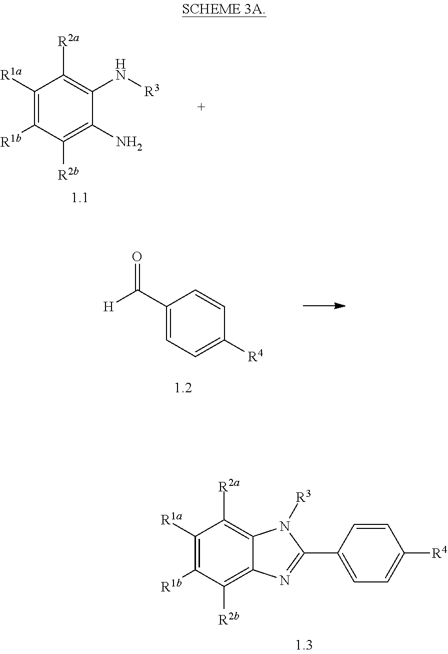

Compounds are represented in generic form, with substituents as noted in compound descriptions elsewhere herein. A more specific example is set forth below.

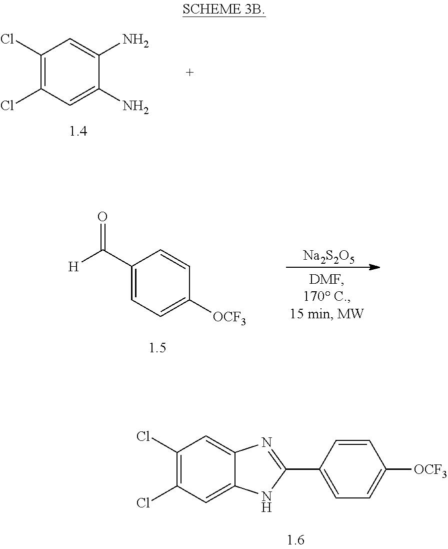 [SMILES: Nc1cc(Cl)c(Cl)cc1N ; O=Cc1ccc(OC(F)(F)F)cc1 ; "Na2S2O5" ; FC(F)(F)Oc1ccc(-c2nc3cc(Cl)c(Cl)cc3[nH]2)cc1]

In one aspect, compounds of type 1.6, and similar compounds, can be prepared according to reaction Scheme 3B above. Thus, compounds of type 1.3 can be prepared by a condensation reaction of an appropriate diamine, e.g., 1.1 as shown above, and an appropriate benzaldehyde, e.g., 1.2 as shown above. Appropriate diamines and appropriate benzaldehydes are commercially available or prepared by methods known to one skilled in the art. The condensation reaction is carried out in the presence of an appropriate base, e.g., sodium metabisulfite, in an appropriate solvent, e.g., dimethylformamide, at an appropriate temperature, e.g., 170° C., for an appropriate period of time, e.g., 15 minutes. As can be appreciated by one skilled in the art, the above reaction provides an example of a generalized approach wherein compounds similar in structure to the specific reactants above (compounds similar to compounds of type 1.1 and 1.2), can be substituted in the reaction to provide substituted benzimidazoles similar to Formula 1.6.

### 4. Route IV

In one aspect, benzimidazole analogs can be prepared as shown below.

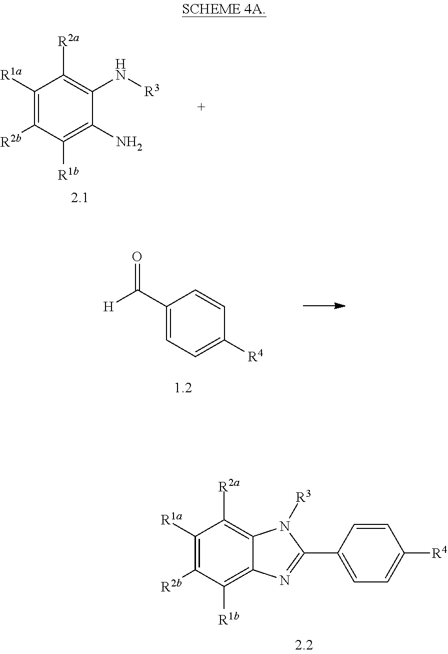

Compounds are represented in generic form, with substituents as noted in compound descriptions elsewhere herein. A more specific example is set forth below.

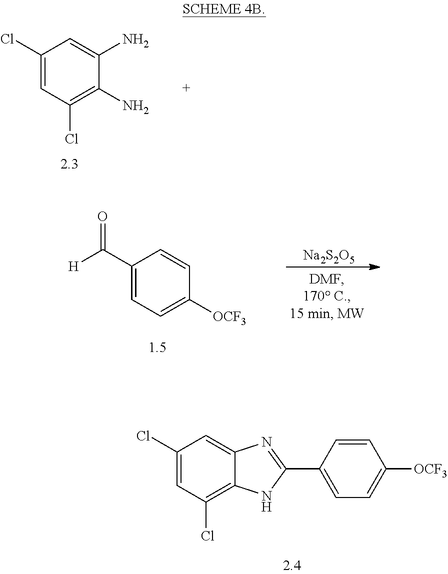 [SMILES: Nc1cc(Cl)cc(Cl)c1N ; O=Cc1ccc(OC(F)(F)F)cc1 ; "Na2S2O5" ; FC(F)(F)Oc1ccc(-c2nc3cc(Cl)cc(Cl)c3[nH]2)cc1]

In one aspect, compounds of type 2.4, and similar compounds, can be prepared according to reaction Scheme 4B above. Thus, compounds of type 2.2 can be prepared by a condensation reaction of an appropriate diamine, e.g., 2.1 as shown above, and an appropriate benzaldehyde, e.g., 1.2 as shown above. Appropriate diamines and appropriate benzaldehydes are commercially available or prepared by methods known to one skilled in the art. The condensation reaction is carried out in the presence of an appropriate base, e.g., sodium metabisulfite, in an appropriate solvent, e.g., dimethylformamide, at an appropriate temperature, e.g., 170° C., for an appropriate period of time, e.g., 15 minutes. As can be appreciated by one skilled in the art, the above reaction provides an example of a generalized approach wherein compounds similar in structure to the specific reactants above (compounds similar to compounds of type 2.1 and 1.2), can be substituted in the reaction to provide substituted benzimidazoles similar to Formula 2.4.

### E. Methods of Using the Compounds

The compounds and pharmaceutical compositions of the invention are useful in treating or controlling disorders associated with Wnt dysfunction, in particular cancers, such as pancreatic cancer, breast cancer, and colon cancer.

Examples of cancers for which the compounds and compositions can be useful in treating, include, but are not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, Polycythemia vera, Lymphoma, Hodgkin's disease, non-Hodgkin's disease, Multiple myeloma, Waldenstrom's macroglobulinemia, Heavy chain disease, Solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma, osteosarcoma, colorectal, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. In a further aspect, the cancer is selected from renal cancer and thyroid cancer.

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a disorder, such as cancer.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disease or condition, such as cancer.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Treatment Methods

The compounds disclosed herein are useful for treating or controlling disorders associated with Wnt dysfunction, in particular cancers, such as pancreatic cancer, breast cancer, and colon cancer. Thus, provided is a method comprising administering a therapeutically effective amount of a composition comprising a disclosed compound to a subject. In a further aspect, the method can be a method for treating a disorder associated with Wnt dysfunction.

### a. Treating a Disorder Associated with Wnt Dysfunction

In one aspect, disclosed are methods of treating a disorder associated with Wnt dysfunction in a mammal, the method comprising the step of administering to the mammal an effective amount of at least one disclosed compound, or a pharmaceutically acceptable salt thereof.

In one aspect, disclosed are methods for the treatment of a disorder associated with Wnt dysfunction in a mammal, the method comprising the step of administering to the mammal an effective amount of at least one compound having a structure.

Examples of disorders associated with Wnt dysfunction include, but are not limited to, cancer, restenosis associated with angioplasty, polycystic kidney disease, aberrant angiogenesis disease, osteoporosis, osteoarthritis, diabetes, non-oncogenic proliferative diseases, rheumatoid arthritis disease, ulcerative colitis, tuberous sclerosis complex, hair loss, and neurodegenerative diseases such as Alzheimer's disease.

In a further aspect, the subject has been diagnosed with a need for treatment of the disorder prior to the administering step.

In a further aspect, the subject is a mammal. In a still further aspect, the mammal is a human.

In a further aspect, the method further comprises the step of identifying a subject in need of treatment of the disorder.

In a further aspect, the disorder is associated with abnormal Wnt signaling activity. In a still further aspect, the disorder is associated with Wnt overexpression.

In a further aspect, the disorder is a cancer. In a still further aspect, the cancer is selected from leukemia, acute leukemia, acute lymphocytic leukemia, acute myelocytic leukemia, myeloblastic, promyelocytic, myelomonocytic, monocytic, erythroleukemia, chronic leukemia, chronic myelocytic (granulocytic) leukemia, chronic lymphocytic leukemia, Polycythemia vera, Lymphoma, Hodgkin's disease, non-Hodgkin's disease, Multiple myeloma, Waldenstrom's macroglobulinemia, Heavy chain disease, Solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, glioblastoma, osteosarcoma, colorectal, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. In yet a further aspect, the cancer is selected from renal cancer and thyroid cancer.

In a further aspect, the method further comprises the step of administering a therapeutically effective amount of at least one agent known to treat a cancer. In a still further aspect, the at least one agent is selected from uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin (Eloxatin®), iressa (gefinitib, Zd1839), XELODA® (capecitabine), Tarceva® (erlotinib), azacitidine (5-Azacytidine; 5-AzaC), temozolomide (Temodar®), gemcitabine (e.g., GEMZAR® (gemcitabine HCl)), and vasostatin.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

In a further aspect, the compound is selected to have activity of less than about 5 μM against a pancreatic cancer stem cell line. In a still further aspect, the pancreatic cancer stem cell line is selected from PANC-1, Suit-2, S2VP10, and L3.6p1.

In a further aspect, the compound has a structure selected from:

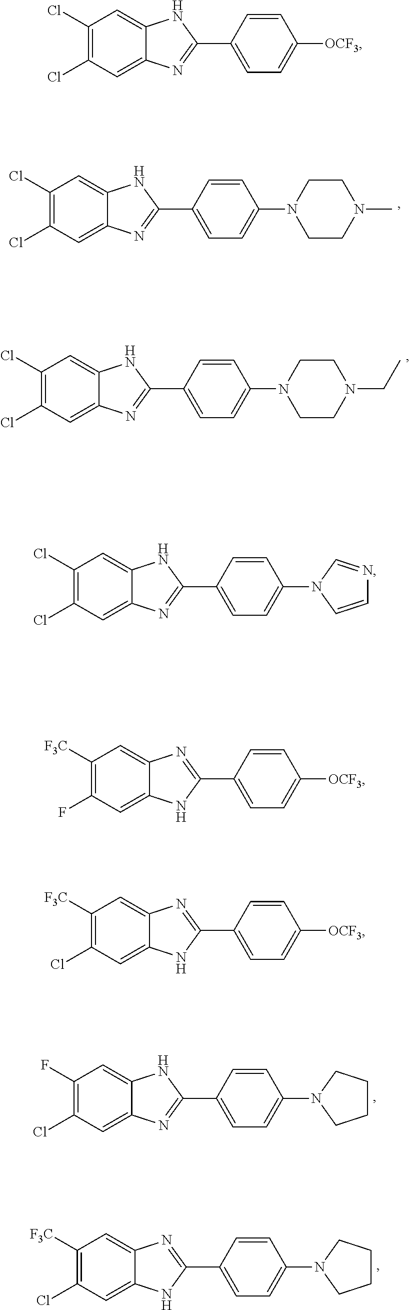

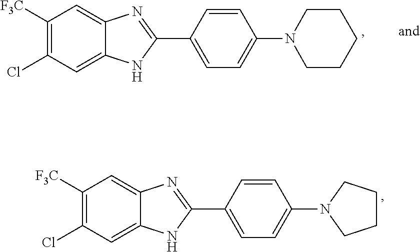 [SMILES: FC(F)(F)c1cc2nc(-c3ccc(N4CCCCC4)cc3)[nH]c2cc1Cl ; FC(F)(F)c1cc2nc(-c3ccc(N4CCCC4)cc3)[nH]c2cc1Cl]

or a pharmaceutically acceptable salt thereof.

In a further aspect, the compound has a structure selected from:

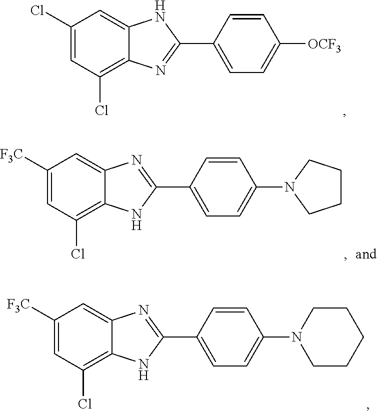 [SMILES: FC(F)(F)Oc1ccc(-c2nc3c(Cl)cc(Cl)cc3[nH]2)cc1 ; FC(F)(F)c1cc(Cl)c2[nH]c(-c3ccc(N4CCCC4)cc3)nc2c1 ; FC(F)(F)c1cc(Cl)c2[nH]c(-c3ccc(N4CCCCC4)cc3)nc2c1]

or a pharmaceutically acceptable salt thereof.

In a further aspect, the compound has a structure selected from:

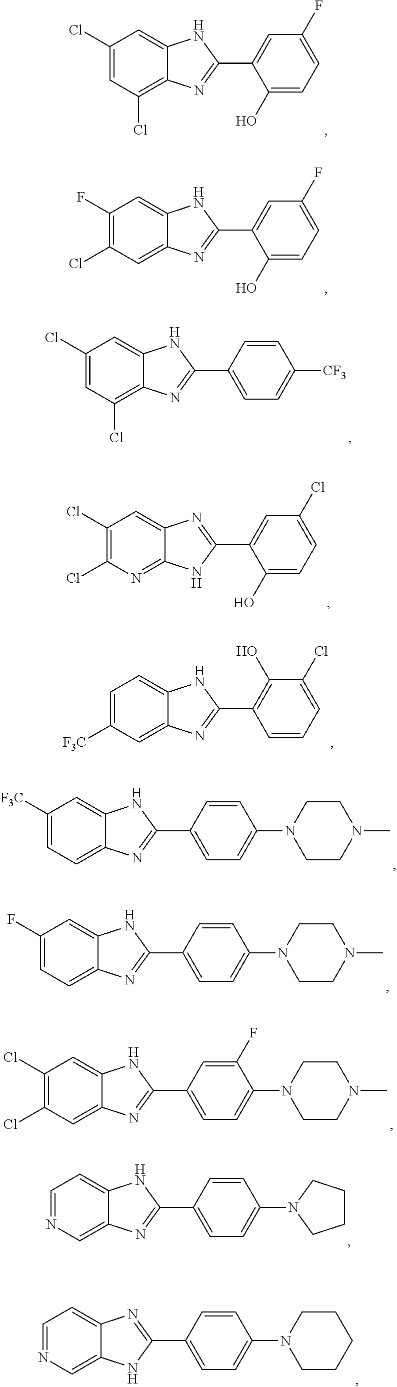

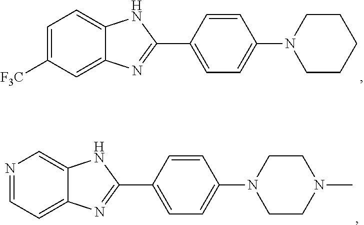 [SMILES: FC(F)(F)c1ccc2[nH]c(-c3ccc(N4CCCCC4)cc3)nc2c1 ; CN1CCN(c2ccc(-c3nc4ccncc4[nH]3)cc2)CC1]

or a pharmaceutically acceptable salt thereof.

### 2. Methods of Inhibiting Wnt Activity in a Mammal

In one aspect, disclosed are methods of inhibiting Wnt activity in a mammal, the method comprising the step of administering to the mammal a therapeutically effective amount of at least one disclosed compound, or a pharmaceutically acceptable salt thereof.

In a further aspect, the compound exhibits inhibition of Wnt activity. In a still further aspect, the compound exhibits a decrease in Wnt activity.

In a further aspect, the compound exhibits inhibition of Wnt activity with an IC₅₀ of less than about 10 μM. In a still further aspect, the compound exhibits inhibition of Wnt activity with an IC₅₀ of less than about 5 μM. In yet a further aspect, the compound exhibits inhibition of Wnt activity with an IC₅₀ of less than about 1 μM. In an even further aspect, the compound exhibits inhibition of Wnt activity with an IC₅₀ of less than about 0.5 μM. In a still further aspect, the compound exhibits inhibition of Wnt activity with an IC₅₀ of less than about 0.1 μM. In yet a further aspect, the compound exhibits inhibition of Wnt activity with an IC₅₀ of less than about 0.05 μM. In an even further aspect, the compound exhibits inhibition of Wnt activity with an IC₅₀ of less than about 0.01 μM. In a still further aspect, the compound exhibits inhibition of Wnt activity with an IC₅₀ of less than about 0.005 μM. In yet a further aspect, the compound exhibits inhibition of Wnt activity with an IC₅₀ of less than about 0.001 μM. In an even further aspect, the compound exhibits inhibition of Wnt activity with an IC₅₀ of less than about 0.0005 μM.

In a further aspect, the subject is a mammal. In a still further aspect, the subject is a human.

In a further aspect, the subject has been diagnosed with a need for treatment of the disorder prior to the administering step. In a still further aspect, the method further comprises the step of identifying a subject in need of treatment of the disorder.

### 3. Methods of Inhibiting Wnt Activity in at Least One Cell

In one aspect, disclosed are methods for inhibiting Wnt activity in at least one cell, the method comprising the step of contacting the at least one cell with an effective amount of at least one disclosed compound, or a pharmaceutically acceptable salt thereof.

In a further aspect, the cell is mammalian. In a still further aspect, the cell is human. In yet a further aspect, the cell has been isolated from a mammal prior to the contacting step.

In a further aspect, contacting is via administration to a mammal.

In a further aspect, the compound is selected to have activity of less than about 5 μM against a pancreatic cancer stem cell line. In a still further aspect, the pancreatic cancer stem cell line is selected from PANC-1, Suit-2, S2VP10, and L3.6p1.

### 4. Use of Compounds

In one aspect, the invention relates to the use of a disclosed compound or a product of a disclosed method. In a further aspect, a use relates to the manufacture of a medicament for the treatment of a disorder associated with Wnt dysfunction in a mammal.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a disorder in a mammal. Also disclosed is the use of a compound for Wnt antagonism. In one aspect, the use is characterized in that the mammal is a human. In one aspect, the use is characterized in that the disorder is a disorder associated with Wnt dysfunction. In one aspect, the disorder associated with Wnt dysfunction is treated by antagonism of Wnt activity in a mammal.

In a further aspect, the use relates to the manufacture of a medicament for the treatment of a disorder associated with Wnt dysfunction in a mammal.

In a further aspect, the use relates to antagonism of Wnt activity in a mammal. In a further aspect, the use relates to modulating Wnt activity in a mammal. In a still further aspect, the use relates to modulating Wnt activity in a cell. In yet a further aspect, the mammal is a human.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a disorder associated with Wnt dysfunction in a mammal. In a further aspect, the disorder is cancer.

### 5. Manufacture of a Medicament

In one aspect, the invention relates to a method for the manufacture of a medicament for treating a disorder associated with Wnt dysfunction in a mammal, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the inhibition of protein and especially Wnt. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the cancer.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

### 6. Kits

In one aspect, the invention relates to a kit comprising a therapeutically effective amount of at least one disclosed compound, or a pharmaceutically acceptable salt thereof, and one or more of: a) at least one agent known to treat a disorder associated with Wnt dysfunction; or b) instructions for treating a disorder associated with Wnt dysfunction.

In a further aspect, the disorder associated with Wnt dysfunction is cancer. In a still further agent the at least one agent is a chemotherapeutic agent. Examples of chemotherapeutic agents include, but are not limited to, alkylating agents such as busulfan, cis-platin, mitomycin C, and carboplatin; antimitotic agents such as colchicine, vinblastine, paclitaxel (e.g., TAXOL®), and docetaxel; topoisomerase I inhibitors such as camptothecin and topotecan; topoisomerase II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea, gemcitabine, capecitabine and thioguanine; antibodies such as HERCEPTIN® and RITUXAN®, as well as other known chemotherapeutics such as photofrin, melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

In a further aspect, the at least one compound and the at least one agent are co-formulated. In a further aspect, the at least one compound and the at least one agent are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

The following preparations and examples are given to enable those skilled in the art to more clearly understand and to practice the present invention. They should not be considered as limiting the scope of the invention, but merely as being illustrative and representative.

### 1. General Experimental Methods

Reagents were purchased from commercial sources and were used as received. Proton nuclear magnetic resonance spectra were obtained on a VARIAN 400 spectrometer at 400 MHz with tetramethylsilane used as an internal reference. The reactions were monitored by Thin-layer chromatography (TLC) on pre-coated silica gel (60F₂₅₄) aluminium plates (0.25 mm) from E. Merck and visualized using UV light (254 nm). Pure samples were dried overnight under high vacuum over P₂O₅ at 78° C. before analyses. The HR-mass spectral data were obtained on an Agilent LC-MSTOF by electrospray ionization (ESI). Purification of compounds were done on an Isco Teledyne Combiflash Rf200 with four channels to carryout sequential purification. Universal Redi Sep solid sample loading pre-packed cartridges (2.5 g silica) were used to absorb crude product and purified on 12 g silica Redi Sep Rf Gold Silica (20-40 μm spherical silica) columns using different solvent gradients.

### 2. Chemistry Experimentals

### a. General Synthesis of Benzimidazoles 1, 3, 5, and 6-8 (Method A)

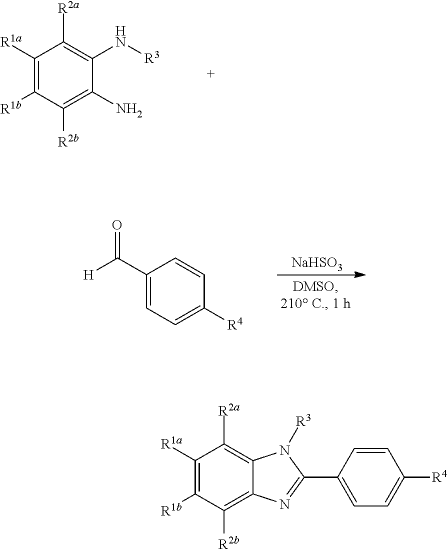

A mixture of an appropriate 1,2-phenylenediamine (1.0 eq), appropriate aldehyde (1.0 eq), and sodium bisulfite (1.0 eq) in 10 mL of DMSO was heated at 210° C. for 1 h. Reaction mixture was concentrated and purified on Biotage purification system using 10-50% hexanes and ethylacetate.

### i. Preparation of Example 1

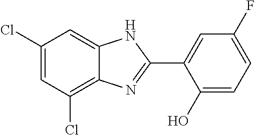 [SMILES: Oc1ccc(F)cc1-c1nc2c(Cl)cc(Cl)cc2[nH]1]

This compound was prepared from 3,5-dichlorobenzene-1,2-diamine (CAS Number: 5233-04-5) and 5-fluoro-2-hydroxybenzaldehyde (CAS Number: 347-54-6) using Method A. Yield 21%. TLC R_{f} 0.50 (Hexane-EtOAc, 2:1). ¹H NMR (DMSO-d₆) δ 12.12 (s, 1H), 7.93 (s, 1H), 7.68 (s, 1H), 7.46 (s, 1H), 7.27 (ddd, J=9.0, 8.1, 3.1 Hz, 1H), 7.07 (dd, J=9.1, 4.7 Hz, 1H). ESI-MS (m/z): 297.0 [M+H]⁺.

### ii. Preparation of Example 3

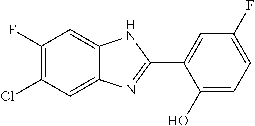 [SMILES: Oc1ccc(F)cc1-c1nc2cc(Cl)c(F)cc2[nH]1]

This compound was prepared from 4-chloro-5-fluorobenzene-1,2-diamine (CAS Number: 139512-70-2) and 5-fluoro-2-hydroxybenzaldehyde (CAS Number: 347-54-6) using Method A. Yield 64%. TLC R_{f} 0.45 (Hexane-EtOAc, 2:1). ¹H NMR (DMSO-d₆) δ 12.60 (s, 1H), 8.54 (s, 1H), 8.22-8.11 (m, 2H), 7.82 (d, J=8.9 Hz, 1H), 7.24-7.09 (m, 1H). ESI-MS (m/z): 281.0 [M+H]⁺.

### iii. Preparation of Example 5

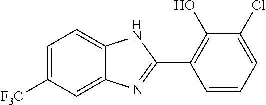 [SMILES: Oc1c(Cl)cccc1-c1nc2cc(C(F)(F)F)ccc2[nH]1]

This compound was prepared from 4-(trifluoromethyl)benzene-1,2-diamine (CAS Number: 368-71-8) and 3-chloro-2-hydroxybenzaldehyde (CAS Number: 1927-94-2) using Method A. Yield 50%. TLC R_{f} 0.45 (Hexane-EtOAc, 2:1). ¹H NMR (DMSO-d₆) δ 8.15 (s, 1H), 8.05 (dd, J=7.9, 1.5 Hz, 1H), 7.85 (s, 1H), 7.67-7.57 (m, 2H), 7.08 (t, J=7.9 Hz, 1H). ESI-MS (m/z): 313.0 [M+H]⁺.

### iv. Preparation of Example 6

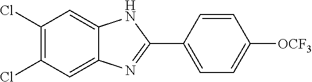 [SMILES: FC(F)(F)Oc1ccc(-c2nc3cc(Cl)c(Cl)cc3[nH]2)cc1]

This compound was prepared from 4,5-dichlorobenzene-1,2-diamine (CAS Number: 5348-42-5) and 4-(trifluoromethoxy)benzaldehyde (CAS Number: 659-28-9) using Method A. Yield 47%. TLC R_{f} 0.45 (Hexane-EtOAc, 2:1). ¹H NMR (DMSO-d₆) δ 13.35 (s, 1H), 8.32-8.24 (m, 2H), 7.88 (s, 2H), 7.58 (dq, J=7.8, 1.1 Hz, 2H). ESI-MS (m/z): 348.0 [M+H]⁺.

### v. Preparation of Example 7

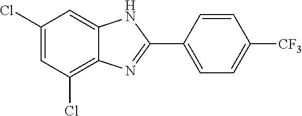 [SMILES: FC(F)(F)c1ccc(-c2nc3c(Cl)cc(Cl)cc3[nH]2)cc1]

This compound was prepared from 3,5-dichlorobenzene-1,2-diamine (CAS Number: 5233-04-5) and 4-(trifluoromethyl)benzaldehyde (CAS Number: 455-19-6) using Method A. Yield 73%. TLC R_{f} 0.45 (Hexane-EtOAc, 2:1). ¹H NMR (DMSO-d₆) δ 13.35 (s, 1H), 8.41 (s, 2H), 7.96 (d, J=8.1 Hz, 2H), 7.78-7.32 (m, 2H). ESI-MS (m/z): 332.0 [M+H]⁺.

### vi. Preparation of Example 8

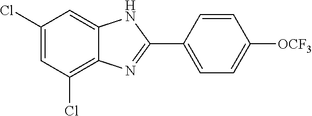 [SMILES: FC(F)(F)Oc1ccc(-c2nc3c(Cl)cc(Cl)cc3[nH]2)cc1]

This compound was prepared from 3,5-dichlorobenzene-1,2-diamine (CAS Number: 5233-04-5) and 4-(trifluoromethoxy)benzaldehyde (CAS Number: 659-28-9) using Method A. Yield 52%. TLC R_{f} 0.45 (Hexane-EtOAc, 2:1). ¹H NMR (DMSO-d₆) δ 8.33 (d, J=8.5 Hz, 4H), 7.58 (d, J=8.5 Hz, 2H). ESI-MS (m/z): 348.0 [M+H]⁺.

### b. General Synthesis of Benzimidazoles 9 and 10 (Method B)

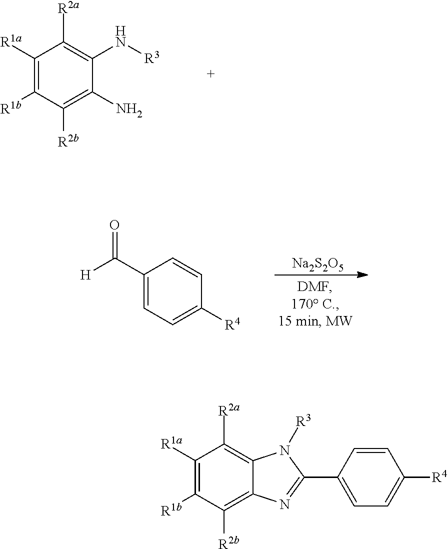

A mixture of an appropriate 1,2-phenylenediamine (1.0 eq), appropriate aldehyde (1.0 eq), and sodium metabisulfite (1.0 eq) in 8 mL of DMF was heated in a CEM microwave at 170° C. for 15 min. Reaction mixture was concentrated and purified on ISCO purification system using 10-50% hexanes and ethylacetate.

### i. Preparation of Example 9

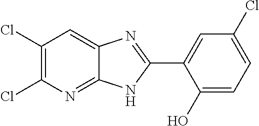 [SMILES: Oc1ccc(Cl)cc1-c1nc2cc(Cl)c(Cl)nc2[nH]1]

This compound was prepared from 5,6-dichloropyridine-2,3-diamine (CAS Number: 97941-89-4) and 5-chloro-2-hydroxybenzaldehyde (CAS Number: 635-93-8) using Method B. Yield 91%. TLC R_{f} 0.35 (Hexane-EtOAc, 2:1). ¹H NMR (DMSO-d₆) δ 8.26 (d, J=2.2 Hz, 1H), 8.17 (dd, J=2.8, 1.2 Hz, 1H), 7.38 (dt, J=8.6, 2.2 Hz, 1H), 7.03 (dd, J=8.9, 1.4 Hz, 1H). ESI-MS (m/z): 315.0 [M+H]⁺.

### ii. Preparation of Example 10

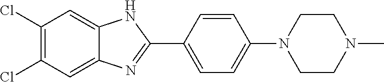 [SMILES: CN1CCN(c2ccc(-c3nc4cc(Cl)c(Cl)cc4[nH]3)cc2)CC1]

This compound was prepared from 4,5-dichlorobenzene-1,2-diamine (CAS Number: 5348-42-5) and 4-(4-methylpiperazin-1-yl)benzaldehyde (CAS Number: 27913-99-1) using Method B. Yield 17%. TLC R_{f} 0.40 (Hexane-EtOAc, 2:1). ¹H NMR (DMSO-d₆) δ 12.88 (s, 1H), 8.00 (dd, J=9.0, 1.8 Hz, 2H), 7.74 (s, 2H), 7.08 (dd, J=9.0, 1.8 Hz, 2H), 3.28 (d, J=5.2 Hz, 4H), 2.46 (t, J=4.9 Hz, 4H), 2.23 (d, J=1.7 Hz, 3H). ESI-MS (m/z): 362.0 [M+H]⁺.

### iii. Preparation of Example 25

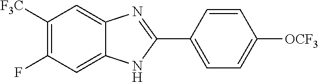 [SMILES: Fc1cc2[nH]c(-c3ccc(OC(F)(F)F)cc3)nc2cc1C(F)(F)F]

This compound was prepared from 4-fluoro-5-(trifluoromethyl)benzene-1,2-diamine and 4-(trifluoromethoxy)benzaldehyde. Yield 48%; TLC R_{f} 0.35 (hexane-EtOAc, 1:1), ¹H NMR (400 MHz, DMSO-d₆) δ 13.58 (s, 1H), 8.34-8.25 (m, 2H), 7.95 (d, J=11.4 Hz, 1H), 7.70 (d, J=11.2 Hz, 1H), 7.59 (dq, J=7.8, 1.0 Hz, 2H). HRMS m/z calcd for C₁₅H₇F₇N₂O+H⁺ [M+H]⁺: 365.0519, found: 365.0517.

### iv. Preparation of Example 26

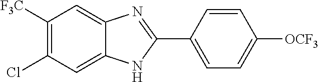 [SMILES: FC(F)(F)Oc1ccc(-c2nc3cc(C(F)(F)F)c(Cl)cc3[nH]2)cc1]

This compound was prepared from 4-chloro-5-(trifluoromethyl)benzene-1,2-diamine and 4-(trifluoromethoxy)benzaldehyde. Yield 61%; TLC R_{f} 0.35 (hexane-EtOAc, 1:1), ¹H NMR (400 MHz, DMSO-d₆) δ 8.34-8.26 (m, 2H), 8.06 (s, 1H), 7.92 (d, J=10.4 Hz, 1H), 7.59 (dq, J=8.9, 1.0 Hz, 2H). HRMS m/z calcd for C₁₅H₇ClF₆N₂O+H⁺ [M+H]⁺: 381.0224, found: 381.0223.

### v. Preparation of Example 27

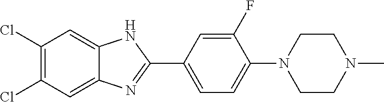 [SMILES: CN1CCN(c2ccc(-c3nc4cc(Cl)c(Cl)cc4[nH]3)cc2F)CC1]

This compound was prepared from 4,5-dichlorobenzene-1,2-diamine and 3-fluoro-4-(4-methylpiperazin-1-yl)benzaldehyde. Yield 37%; TLC R_{f} 0.35 (hexane-EtOAc, 1:1), ¹H NMR (400 MHz, DMSO-d₆) δ 13.11 (s, 1H), 7.93-7.86 (m, 2H), 7.84 (d, J=2.0 Hz, 1H), 7.82-7.77 (m, 2H), 7.17 (t, J=8.9 Hz, 1H), 3.13 (q, J=6.4, 4.9 Hz, 5H), 2.46 (d, J=4.7 Hz, 2H), 2.22 (s, 3H). HRMS m/z calcd for C₁₈H₁₇Cl₂FN₄+H⁺ [M+H]⁺: 379.0887, found: 379.0880.

### vi. Preparation of Example 28

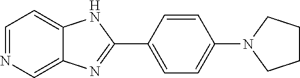 [SMILES: c1cc2[nH]c(-c3ccc(N4CCCC4)cc3)nc2cn1]

This compound was prepared from pyridine-3,4-diamine and 4-(pyrrolidin-1-yl)benzaldehyde. Yield 43%; TLC R_{f} 0.35 (hexane-EtOAc, 1:1), ¹H NMR (400 MHz, DMSO-d₆) δ 12.87 (s, 1H), 8.79 (s, 1H), 8.21 (d, J=5.5 Hz, 1H), 8.00 (d, J=8.7 Hz, 2H), 7.45 (s, 1H), 6.71-6.62 (m, 2H), 3.33-3.29 (m, 4H), 2.03-1.91 (m, 4H). HRMS m/z calcd for C₁₆H₁₆N₄+H⁺ [M+H]⁺: 265.1447, found: 265.1444.

### 3. Biology Experimentals

Compounds were assessed for inhibition of Wnt signaling and cell viability using the following procedures.

### a. Cell Lines

Breast cancer SUM159 was obtained from Asterand and grown according to supplier's recommendation. All other cell lines were obtained from ATCC and cultured in standard cell culture conditions with complete cell culture medium containing 10% FBS.

### b. Wnt Signaling Reporter Assay

HEK293 cells were plated into 24-well plates. After overnight culture, the cells were transiently transfected with 0.06 μg of the Super8XTOPFlash luciferase construct, 0.06 μg of β-galactosidase-expressing vector, and 0.06 μg of pCS-Myc-hLRP6 or control vector. After 24 h incubation, cells were treated with each individual compound in triplicate for 24 h. Cells were then lysed, and the luciferase and β-galactosidase activities were determined by the luciferase assay system (Promega) and β-galactosidase assay system (Promega), respectively. The luciferase activity was normalized to the β-galactosidase activity.

### c. In Vitro Cell Viability Assay

Cancer cells were seeded into optically clear tissue culture-treated black 96-well plates (for adherent cells) or low-attachment 96-well plates (for cancer stem cells) at 2,000-5000 cells/50 μL of media per well. The cells were treated with Wnt inhibitors at 0.1 to 10 μM for 72 h. Cell viability was measured by the Cell Titer Glo Assay from Promega.

### 4. Characterization of Exemplary Compounds

The compounds below in Table 1 were synthesized with methods identical or analogous to those described herein. The requisite starting materials were commercially available, described in the literature, or readily synthesized by one skilled in the art of organic synthesis.

TABLE 1

| No. | Structure | Name |
|---|---|---|
| 1 | 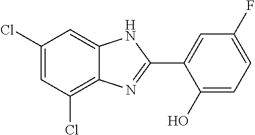 [SMILES: Oc1ccc(F)cc1-c1nc2c(Cl)cc(Cl)cc2[nH]1] | 2-(4,6-Dichloro-1H- benzo[d]imidazol-2-yl)-4- fluorophenol |
| 3 | 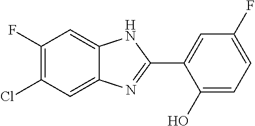 [SMILES: Oc1ccc(F)cc1-c1nc2cc(Cl)c(F)cc2[nH]1] | 2-(5-Chloro-6-fluoro-1H- benzo[d]imidazol-2-yl)-4- fluorophenol |
| 5 | 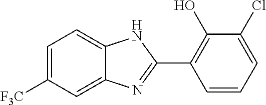 [SMILES: Oc1c(Cl)cccc1-c1nc2cc(C(F)(F)F)ccc2[nH]1] | 2-Chloro-6-(5- (trifluoromethyl)-1H- benzo[d]imidazol-2-yl)phenol |
| 6 | 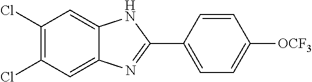 [SMILES: FC(F)(F)Oc1ccc(-c2nc3cc(Cl)c(Cl)cc3[nH]2)cc1] | 5,6-Dichloro-2-(4- (trifluoromethoxy)phenyl)-1H- benzo[d]imidazole |
| 7 | 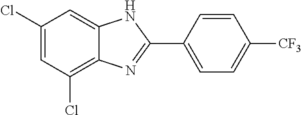 [SMILES: FC(F)(F)c1ccc(-c2nc3c(Cl)cc(Cl)cc3[nH]2)cc1] | 4,6-Dichloro-2-(4- (trifluoromethyl)phenyl)-1H- benzo[d]imidazole |
| 8 | 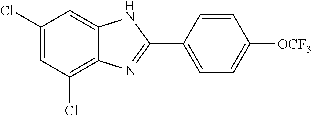 [SMILES: FC(F)(F)Oc1ccc(-c2nc3c(Cl)cc(Cl)cc3[nH]2)cc1] | 4,6-Dichloro-2-(4- (trifluoromethoxy)phenyl)-1H- benzo[d]imidazole |
| 9 | 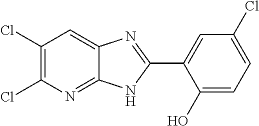 [SMILES: Oc1ccc(Cl)cc1-c1nc2cc(Cl)c(Cl)nc2[nH]1] | 4-Chloro-2-(5,6-dichloro-3H- imidazo[4,5-b]pyridin-2- yl)phenol |
| 10 | 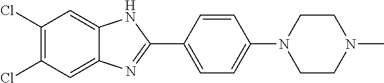 [SMILES: CN1CCN(c2ccc(-c3nc4cc(Cl)c(Cl)cc4[nH]3)cc2)CC1] | 5,6-Dichloro-2-(4-(4- methylpipcrazin-1-yl)phenyl)- 1H-benzo[d]imidazole |
| 11 | 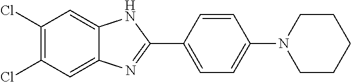 [SMILES: Clc1cc2nc(-c3ccc(N4CCCCC4)cc3)[nH]c2cc1Cl] | 5,6-dichloro-2-(4-(piperidin-1- yl)phenyl)-1H- benzo[d]imidazole |
| 12 | 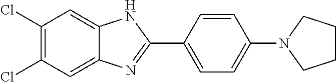 [SMILES: Clc1cc2nc(-c3ccc(N4CCCC4)cc3)[nH]c2cc1Cl] | 5,6-dichloro-2-(4-(pyrrolidin-1- yl)phenyl)-1H- benzo[d]imidazole |
| 13 | 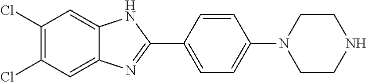 [SMILES: Clc1cc2nc(-c3ccc(N4CCNCC4)cc3)[nH]c2cc1Cl] | 5,6-dichloro-2-(4-(piperazin-1- yl)phenyl)-1H- benzo[d]imidazole |
| 14 | 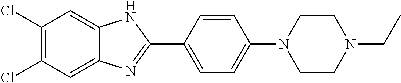 [SMILES: CCN1CCN(c2ccc(-c3nc4cc(Cl)c(Cl)cc4[nH]3)cc2)CC1] | 5,6-dichloro-2-(4-(4- ethylpiperazin-1-yl)phenyl)-1H- benzo[d]imidazole |
| 15 | 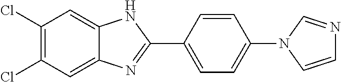 [SMILES: Clc1cc2nc(-c3ccc(-n4ccnc4)cc3)[nH]c2cc1Cl] | 2-(4-(1H-imidazol-1- yl)phenyl)-5,6-dichloro-1H- benzo[d]imidazole |
| 16 | 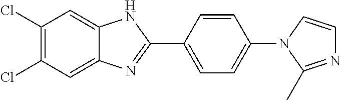 [SMILES: Cc1nccn1-c1ccc(-c2nc3cc(Cl)c(Cl)cc3[nH]2)cc1] | 5,6-dichloro-2-(4-(2-methyl- 1H-imidazol-1-yl)phenyl)-1H- benzo[d]imidazole |
| 17 | 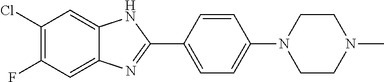 [SMILES: CN1CCN(c2ccc(-c3nc4cc(F)c(Cl)cc4[nH]3)cc2)CC1] | 6-chloro-5-fluoro-2-(4-(4- methylpiperazin-1-yl)phenyl)- 1H-benzo[d]imidazole |
| 18 | 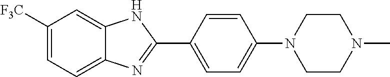 [SMILES: CN1CCN(c2ccc(-c3nc4ccc(C(F)(F)F)cc4[nH]3)cc2)CC1] | 2-(4-(4-methylpiperazin-1- yl)phenyl)-6-(trifluoromethyl)- 1H-benzo[d]imidazole |
| 19 | 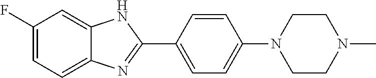 [SMILES: CN1CCN(c2ccc(-c3nc4ccc(F)cc4[nH]3)cc2)CC1] | 6-fluoro-2-(4-(4- methylpiperazin-1-yl)phenyl)- 1H-benzo[d]imidazole |
| 20 | 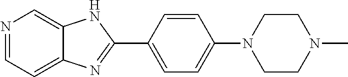 [SMILES: CN1CCN(c2ccc(-c3nc4ccncc4[nH]3)cc2)CC1] | 2-(4-(4-methylpiperazin-1- yl)phenyl)-3H-imidazo[4,5- c]pyridine |
| 21 | 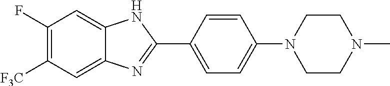 [SMILES: CN1CCN(c2ccc(-c3nc4cc(C(F)(F)F)c(F)cc4[nH]3)cc2)CC1] | 6-fluoro-2-(4-(4- methylpiperazin-1-yl)phenyl)- 5-(trifluoromethyl)-1H- benzo[d]imidazole |
| 22 | 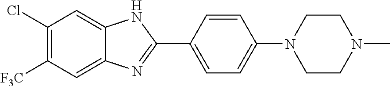 [SMILES: CN1CCN(c2ccc(-c3nc4cc(C(F)(F)F)c(Cl)cc4[nH]3)cc2)CC1] | 6-chloro-2-(4-(4- methylpiperazin-1-yl)phenyl)- 5-(trifluoromethyl)-1H- benzo[d]imidazole |
| 23 | 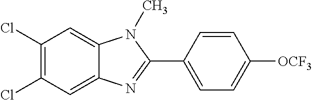 [SMILES: Cn1c(-c2ccc(OC(F)(F)F)cc2)nc2cc(Cl)c(Cl)cc21] | 5,6-dichloro-1-methyl-2-(4- (trifluoromethoxy)phenyl)-1H- benzo[d]imidazole |
| 24 | 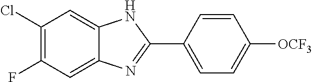 [SMILES: Fc1cc2nc(-c3ccc(OC(F)(F)F)cc3)[nH]c2cc1Cl] | 6-chloro-5-fluoro-2-(4- (trifluoromethoxy)phenyl)-1H- benzo[d]imidazole |
| 25 | 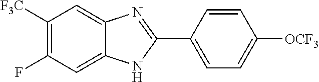 [SMILES: Fc1cc2[nH]c(-c3ccc(OC(F)(F)F)cc3)nc2cc1C(F)(F)F] | 6-fluoro-2-(4- (trifluoromethoxy)phenyl)-5- (trifluoromethyl)-1H- benzo[d]imidazole |
| 26 | 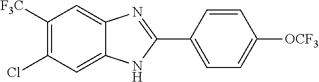 [SMILES: FC(F)(F)Oc1ccc(-c2nc3cc(C(F)(F)F)c(Cl)cc3[nH]2)cc1] | 6-chloro-2-(4- (trifluoromethoxy)phenyl)-5- (trifluoromethyl)-1H- benzo[d]imidazole |
| 27 | 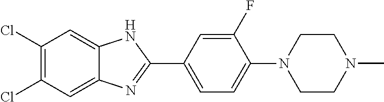 [SMILES: CN1CCN(c2ccc(-c3nc4cc(Cl)c(Cl)cc4[nH]3)cc2F)CC1] | 5,6-dichloro-2-(3-fluoro-4-(4- methylpiperazin-1-yl)phenyl)- 1H-benzo[d]imidazole |
| 28 | 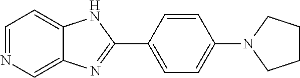 [SMILES: c1cc2[nH]c(-c3ccc(N4CCCC4)cc3)nc2cn1] | 2-(4-(pyrrolidin-1-yl)phenyl)- 1H-imidazo[4,5-c]pyridine |
| 29 | 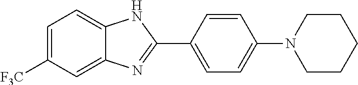 [SMILES: FC(F)(F)c1ccc2[nH]c(-c3ccc(N4CCCCC4)cc3)nc2c1] | 2-(4-(piperidin-1-yl)phenyl)-5- (trifluoromethyl)-1H- benzo[d]imidazole |
| 30 | 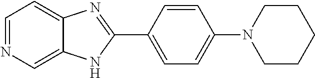 [SMILES: c1cc2nc(-c3ccc(N4CCCCC4)cc3)[nH]c2cn1] | 2-(4-(piperidin-1-yl)phenyl)- 3H-imidazo[4,5-c]pyridine |
| 31 | 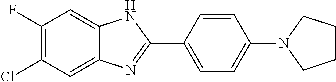 [SMILES: Fc1cc2[nH]c(-c3ccc(N4CCCC4)cc3)nc2cc1Cl] | 5-chloro-6-fluoro-2-(4- (pyrrolidin-1-yl)phenyl)-1H- benzo[d]imidazole |
| 32 | 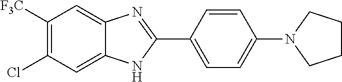 [SMILES: FC(F)(F)c1cc2nc(-c3ccc(N4CCCC4)cc3)[nH]c2cc1Cl] | 6-chloro-2-(4-(pyrrolidin-1- yl)phenyl)-5-(trifluoromethyl)- 1H-benzo[d]imidazole |
| 33 | 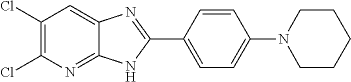 [SMILES: Clc1cc2nc(-c3ccc(N4CCCCC4)cc3)[nH]c2nc1Cl] | 5,6-dichloro-2-(4-(piperidin-1- yl)phenyl)-3H-imidazo[4,5- b]pyridine |
| 34 | 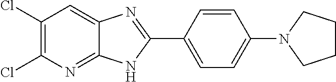 [SMILES: Clc1cc2nc(-c3ccc(N4CCCC4)cc3)[nH]c2nc1Cl] | 5,6-dichloro-2-(4-(pyrrolidin-1- yl)phenyl)-3H-imidazo[4,5- b]pyridine |
| 35 | 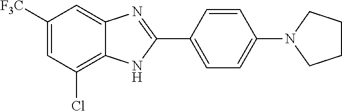 [SMILES: FC(F)(F)c1cc(Cl)c2[nH]c(-c3ccc(N4CCCC4)cc3)nc2c1] | 7-chloro-2-(4-(pyrrolidin-1- yl)phenyl)-5-(trifluoromethyl)- 1H-benzo[d]imidazole |
| 36 | 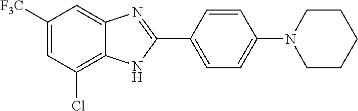 [SMILES: FC(F)(F)c1cc(Cl)c2[nH]c(-c3ccc(N4CCCCC4)cc3)nc2c1] | 7-chloro-2-(4-(piperidin-1- yl)phenyl)-5-(trifluoromethyl)- 1H-benzo[d]imidazole |
| 37 | 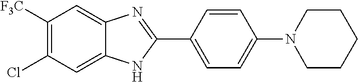 [SMILES: FC(F)(F)c1cc2nc(-c3ccc(N4CCCCC4)cc3)[nH]c2cc1Cl] | 6-chloro-2-(4-(piperidin-1- yl)phenyl)-5-(trifluoromethyl)- 1H-benzo[d]imidazole |
| 38 | 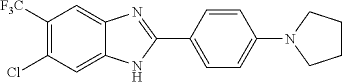 [SMILES: FC(F)(F)c1cc2nc(-c3ccc(N4CCCC4)cc3)[nH]c2cc1Cl] | 6-chloro-2-(4-(pyrrolidin-1- yl)phenyl)-5-(trifluoromethyl)- 1H-benzo[d]imidazole |

### 5. Evaluation of Inhibitory Activity Against Wnt Signaling

Table 2 below illustrates the effects of the disclosed compounds on Wnt signaling in LRP6-expressing HEK293 cells.

TABLE 2

| No. | IC₅₀ (μM) |
|---|---|
| 1 | 1.34 |
| 3 | 2.80 |
| 5 | 2.77 |
| 6 | 0.19 |
| 7 | 6.01 |
| 8 | 7.92 |
| 9 | 1.85 |
| 10 | 0.013 |
| 11 | 0.00095 |
| 12 | 0.00093 |
| 18 | Inactive |
| 19 | Inactive |
| 20 | Inactive |
| 23 | Inactive |
| 24 | Inactive |
| 25 | Inactive |
| 26 | Inactive |
| 27 | 0.00467 |
| 28 | 10.45 |
| 29 | Inactive |
| 30 | Inactive |
| 31 | 0.33 |
| 32 | Inactive |
| 33 | Inactive |
| 34 | Inactive |
| 35 | Inactive |
| 36 | Inactive |
| 37 | Inactive |
| 38 | Inactive |

### 6. Evaluation of Effects on Pancreatic Cancer Cell Viability

Table 3 below illustrates the cell viability of pancreatic cancer cell lines.

TABLE 3

| | Cell viability (IC₅₀, μM) | | | | | | | |
|---|---|---|---|---|---|---|---|---|
| | PANC-1 | | Suit-2 | | S2VP10 | | L3.6pl | |
| No. | Adherent* | CSC** | Adherent | CSC | Adherent | CSC | Adherent | CSC |
| 1 | 1.74 | 0.21 | 9.13 | 2.84 | 1.62 | 0.92 | — | — |
| 3 | 2.32 | 0.41 | 3.76 | 1.81 | 1.1 | 0.33 | — | — |
| 5 | 1.33 | 0.18 | 3.16 | 1.15 | 1.04 | 0.21 | — | — |
| 6 | 3.44 | 0.57 | 2.12 | 0.56 | 2.57 | 0.59 | — | 0.93 |
| 7 | 4.47 | 0.3 | 5.21 | 0.61 | 3.88 | 0.77 | 0.57 | 0.66 |
| 8 | >10 | 1.06 | 8.92 | 0.84 | 4.86 | 0.77 | 1.19 | 0.62 |
| 9 | — | — | >10 | >10 | 2.24 | 0.87 | — | — |
| 10 | — | — | 5.26 | 1.34 | 4.76 | 0.91 | — | — |
| 11 | — | — | 0.97 | >10 | 1.68 | >10 | — | — |
| 12 | — | — | 2.10 | >10 | 2.58 | >10 | — | — |
| 13 | — | — | >10 | >10 | >10 | >10 | — | — |
| 14 | — | — | >10 | 1.23 | >10 | 1.33 | — | — |
| 15 | — | — | >10 | 1.81 | >10 | 1.34 | — | — |
| 16 | — | — | >10 | >10 | >10 | >10 | — | — |
| 18 | — | — | >10 | 0.76 | 7.16 | 1.52 | — | — |
| 19 | — | — | 1.34 | 0.33 | 0.79 | 0.35 | — | — |
| 20 | — | — | 0.29 | 0.21 | 0.14 | 0.18 | — | — |
| 25 | — | — | 5.63 | 2.05 | 7.20 | 2.77 | — | — |
| 26 | — | — | 6.44 | 0.86 | 6.92 | 0.95 | — | — |
| 27 | — | — | 8.92 | 1.99 | 8.31 | 2.91 | — | — |
| 28 | — | — | 0.16 | 0.15 | 0.11 | 0.08 | — | — |
| 29 | — | — | 7.56 | 1.42 | >10 | 1.08 | — | — |
| 30 | — | — | 0.08 | 0.07 | 0.06 | — | — | — |
| 31 | — | — | 0.95 | 0.94 | 0.51 | 0.75 | — | — |
| 32 | — | — | 3.87 | 1.10 | 5.20 | 0.60 | — | — |
| 33 | — | — | >10 | >10 | >10 | >10 | — | — |
| 34 | — | — | >10 | >10 | >10 | >10 | — | — |
| 35 | — | — | 9.06 | 0.24 | >10 | 0.15 | — | — |
| 36 | — | — | 8.66 | 2.53 | >10 | 2.95 | — | — |
| 37 | — | — | 5.33 | 0.55 | 9.25 | — | — | — |
| 38 | — | — | 3.87 | 1.10 | 5.20 | 0.60 | — | — |

*Cancer cells were cultured in standard cell culture conditions (attached cells in complete medium containing 10% FBS).
**Cancer cells were cultured in conditions designed to enrich for cancer stem cells (CSC), including serum free medium (RPMI supplemented with 20 ng/ml EGF, 10 ng/ml basic FGF, 25 μg/ml insulin, 5 μg/ml transferrin, 5 ng/ml sodium selenite, 16 μg/ml putrescene, and 7.3 ng/ml progesterone) in non-adherent cell culture plates.

### 7. Evaluation of Effects on Colorectal Cancer Cell Viability

Table 4 below illustrates the effects of the disclosed compounds on cell viability of colorectal cancer cell lines.

TABLE 4

| Cell viability (IC₅₀, μM) | |
|---|---|
| HCT116 | HT-29 |

| No. | Adherent | CSC | Adherent | CSC |
|---|---|---|---|---|
| 3 | 1.61 | 0.18 | 6.47 | 3.09 |
| 6 | 3.84 | 1.61 | 4.38 | 0.86 |
| 9 | 2.09 | 0.51 | 9.76 | 8.95 |
| 10 | 11.24 | 20.95 | 3.29 | 0.13 |
| 11 | 1.01 | 0.57 | 0.67 | 0.04 |
| 12 | 1.18 | 2.68 | 0.84 | 0.51 |
| 27 | 0.83 | 1.76 | 0.14 | — |
| 28 | 0.11 | 0.08 | 0.06 | 0.05 |
| 31 | 0.58 | 0.16 | 0.59 | 0.04 |

### 8. Evaluation of Effects on Breast Cancer Cell Viability

Table 5 below illustrates the effects of the disclosed compounds on cell viability of breast cancer cell lines.

TABLE 5

| | Cell viability (IC₅₀, μM) | | | | | | | |
|---|---|---|---|---|---|---|---|---|
| | 2LMP | | HCC1143 | | MDA-MB-468 | | SUM159 | |
| No. | Adherent | CSC | Adherent | CSC | Adherent | CSC | Adherent | CSC |
| 5 | 1.15 | 0.92 | 1.19 | 0.50 | 1.41 | 0.17 | 0.50 | 0.34 |
| 6 | 3.84 | 2.90 | 3.18 | 1.30 | 3.83 | 0.33 | 1.67 | 1.17 |
| 7 | 16.79 | 12.95 | 4.50 | 0.70 | 1.29 | 0.16 | 1.13 | 0.90 |

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A compound having a structure represented by a formula: 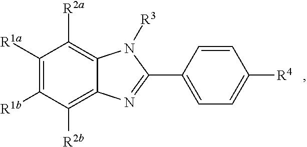
wherein each of R^{1a} and R^{1b} is independently selected from halogen, —CF₃, and —OR²⁰,
wherein each occurrence of R²⁰, when present, is independently selected from hydrogen, C1-C4 alkyl, C1-C4 monohaloalkyl, and C1-C4 polyhaloalkyl;
wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy;
wherein R³ is selected from hydrogen and C1-C4 alkyl;
wherein R⁴ is selected from —OCF₃ and a structure represented by a formula selected from: 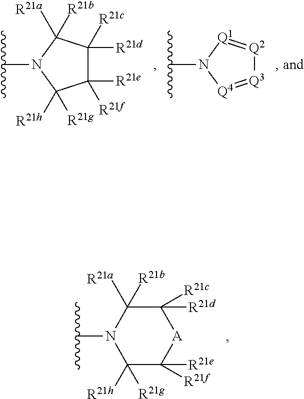
wherein A, when present, is selected from O, NR³⁰, and CR^{31a}R^{31b};
wherein R³⁰, when present, is selected from hydrogen and C1-C4 alkyl;
wherein each of R^{31a} and R^{31b}, when present, is independently selected from hydrogen and C1-C4 alkyl;
wherein each of Q¹, Q², Q³, and Q⁴, when present, is independently selected from CR³⁰ and N; and
wherein each of R^{21a}, R^{21b}, R^{21c}, R^{21d}, R^{21e}, R^{21f}, R^{21g}, and R^{21h}, when present, is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy,
or a pharmaceutically acceptable salt thereof.

2. The compound of claim 1, wherein R⁴ is a structure represented by a formula selected from:

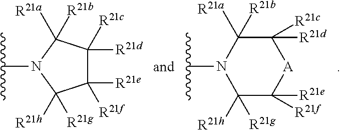

3. The compound of claim 1, wherein the compound has a structure represented by a formula: 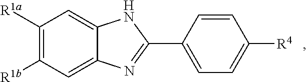
or a pharmaceutically acceptable salt thereof.

4. The compound of claim 1, wherein the compound has a structure represented by a formula: 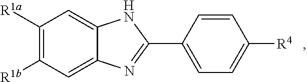
wherein R⁴ is a structure represented by a formula selected from: 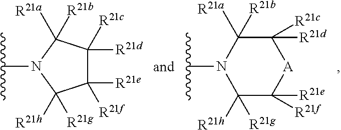
or a pharmaceutically acceptable salt thereof.

5. The compound of claim 1, wherein the compound has a structure selected from: 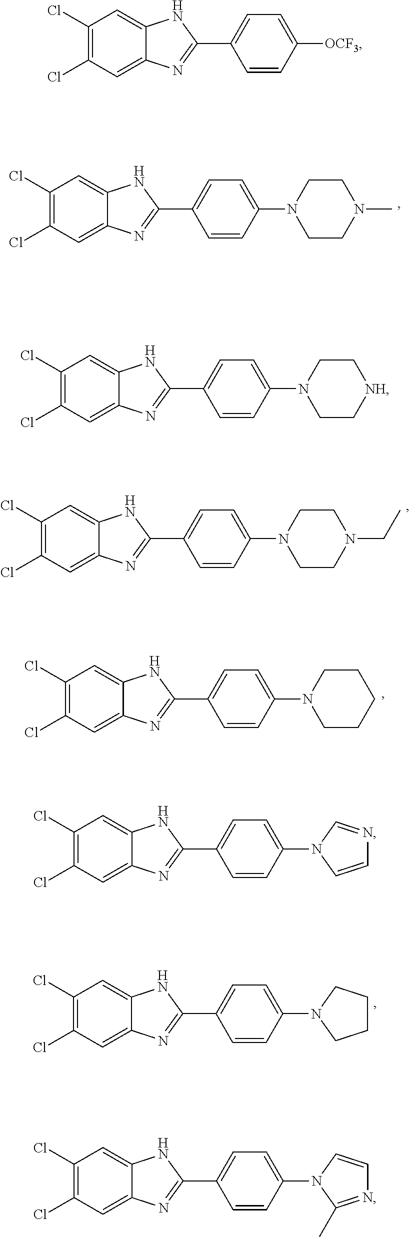 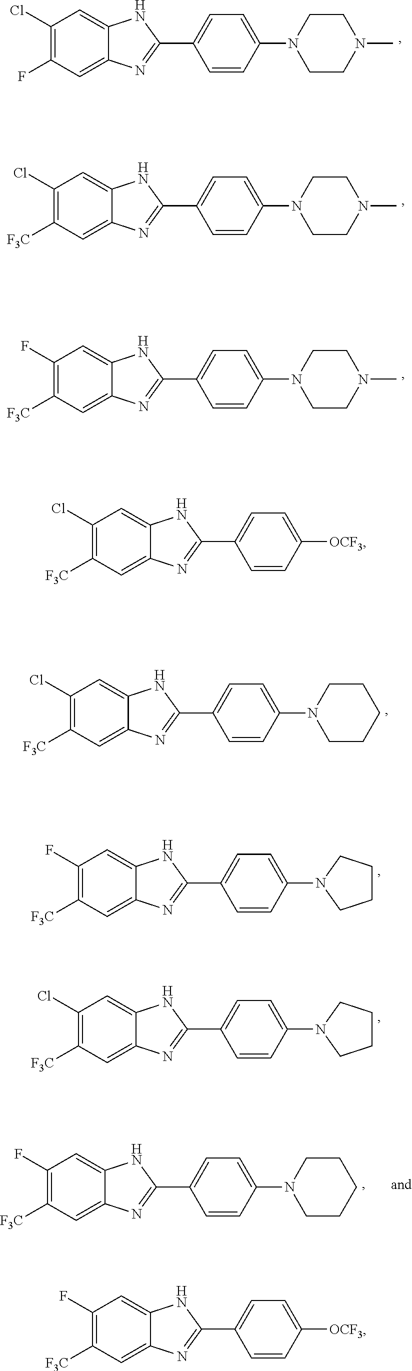
or a pharmaceutically acceptable salt thereof.

6. The compound of claim 1, wherein the compound has a structure selected from: 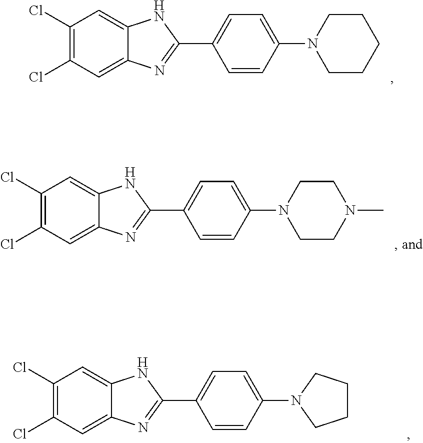 [SMILES: Clc1cc2nc(-c3ccc(N4CCCCC4)cc3)[nH]c2cc1Cl ; CN1CCN(c2ccc(-c3nc4cc(Cl)c(Cl)cc4[nH]3)cc2)CC1 ; Clc1cc2nc(-c3ccc(N4CCCC4)cc3)[nH]c2cc1Cl]
or a pharmaceutically acceptable salt thereof.

7. A method of treating a disorder associated with Wnt dysfunction in a subject, the method comprising administering to the subject an effective amount of at least one compound of claim 1, or a pharmaceutically acceptable salt thereof.

8. The method of claim 7, wherein the disorder is cancer.

9. The method of claim 7, wherein the compound is selected to have activity of less than about 5 μM against a pancreatic cancer stem cell line.

10. The method of claim 9, wherein the pancreatic cancer stem cell line is selected from PANC-1, Suit-2, S2VP10, and L3.6p1.

11. The method of claim 9, wherein the compound has a structure selected from: 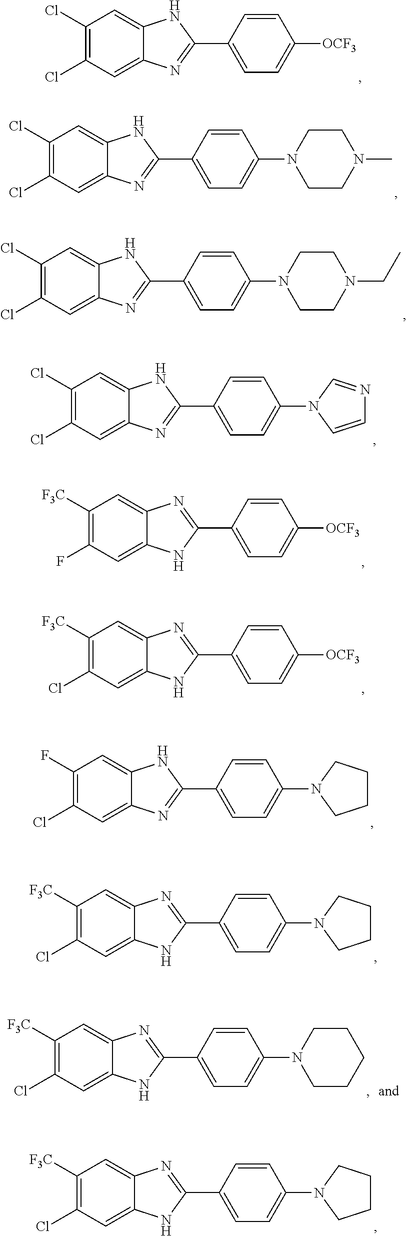
or a pharmaceutically acceptable salt thereof.

12. A compound having a structure represented by a formula: 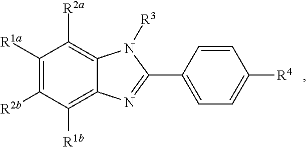
wherein each of R^{1a} and R^{1b} is independently selected from halogen, —CF₃, and —OR²⁰;
wherein each occurrence of R²⁰, when present, is independently selected from hydrogen, C1-C4 alkyl, C1-C4 monohaloalkyl, and C1-C4 polyhaloalkyl;
wherein each of R^{2a} and R^{2b} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy;
wherein R³ is selected from hydrogen and C1-C4 alkyl;
wherein R⁴ is selected from —OCF₃ and a structure represented by a formula selected from: 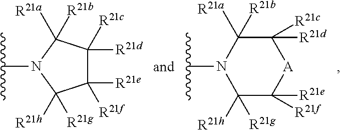
wherein A, when present, is selected from O, NR³⁰, and CR^{31a}R^{31b};
wherein R³⁰, when present, is selected from hydrogen and C1-C4 alkyl;
wherein each of R^{31a} and R^{31b}, when present, is independently selected from hydrogen and C1-C4 alkyl; and
wherein each of R^{21a}, R^{21b}, R^{21c}, R^{21d}, R^{21e}, R^{21f}, R^{21g}, and R^{21h} is independently selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy,
or a pharmaceutically acceptable salt thereof.

13. The compound of claim 12, wherein the compound has a structure selected from: 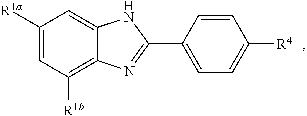
or a pharmaceutically acceptable salt thereof.

14. The compound of claim 12, wherein the compound has a structure selected from: 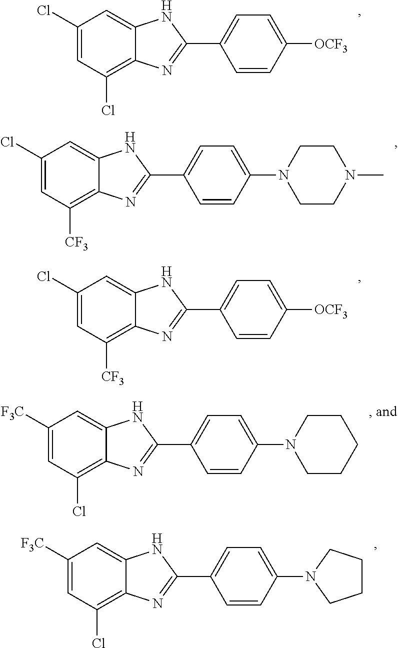
or a pharmaceutically acceptable salt thereof.

15. A method of treating a disorder associated with Wnt dysfunction in a subject, the method comprising administering to the subject an effective amount of at least one compound of claim 12, or a pharmaceutically acceptable salt thereof.

16. The method of claim 15, wherein the disorder is cancer.

17. The method of claim 15, wherein the compound is selected to have activity of less than about 5 μM against a pancreatic cancer stem cell line.

18. The method of claim 17, wherein the pancreatic cancer stem cell line is selected from PANC-1, Suit-2, S2VP10, and L3.6p1.

19. The method of claim 17, wherein the compound has a structure selected from: 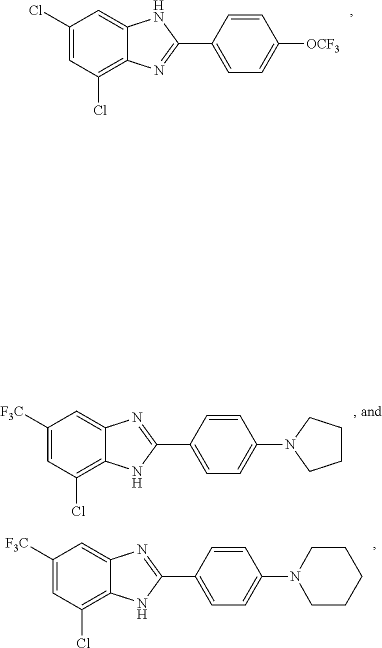
or a pharmaceutically acceptable salt thereof.

20. A compound having a structure selected from: 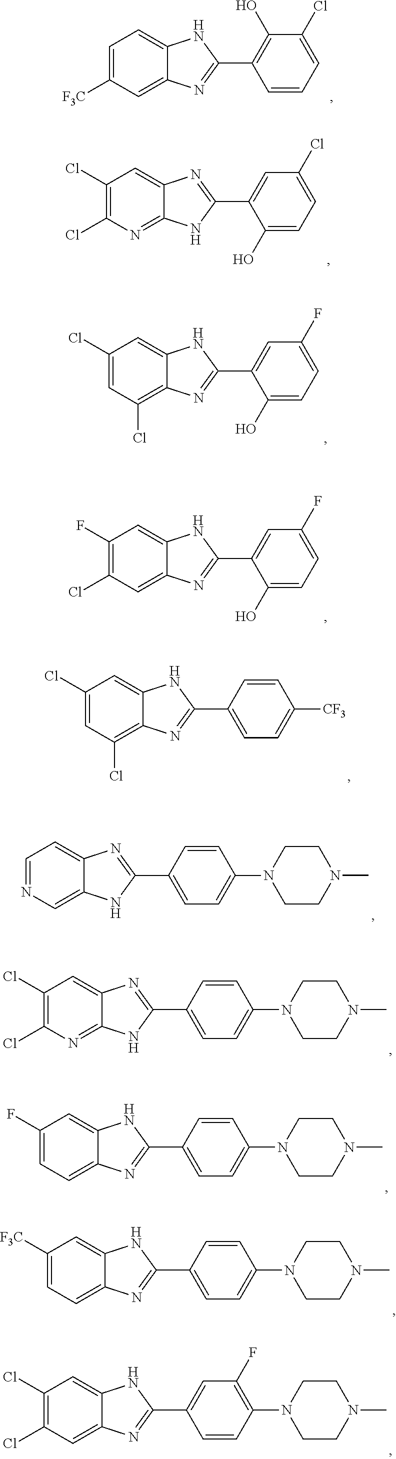 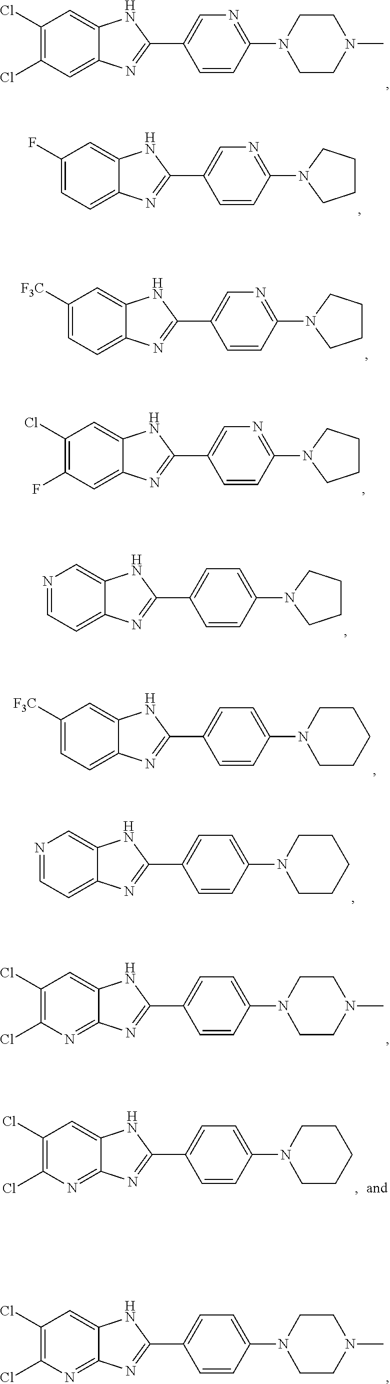
or a pharmaceutically acceptable salt thereof.

21. A method of treating a disorder associated with Wnt dysfunction in a subject, the method comprising administering to the subject an effective amount of at least one compound of claim 20, or a pharmaceutically acceptable salt thereof.

22. The method of claim 21, wherein the disorder is cancer.

23. The method of claim 21, wherein the compound is selected to have activity of less than about 5 μM against a pancreatic cancer stem cell line.

24. The method of claim 23, wherein the pancreatic cancer stem cell line is selected from PANC-1, Suit-2, S2VP10, and L3.6p1.

25. The method of claim 23, wherein the compound has a structure selected from: 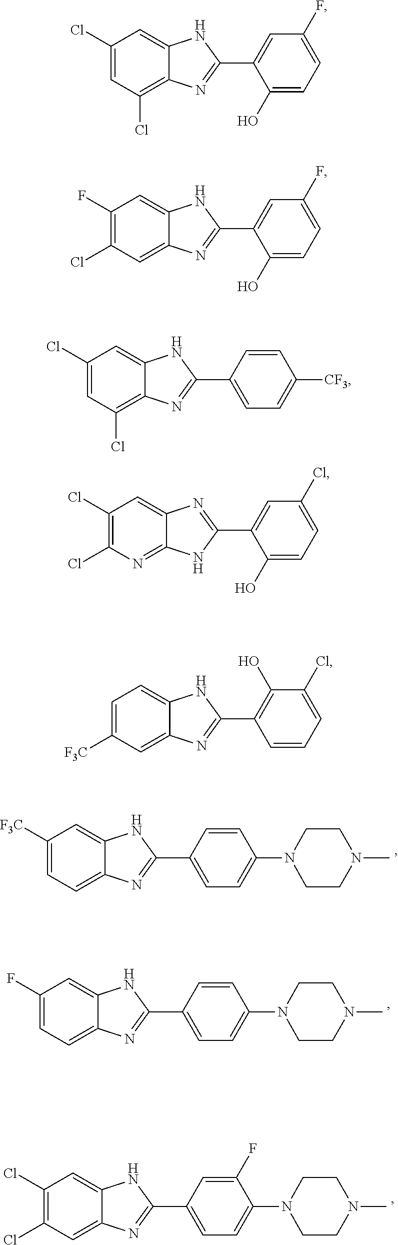 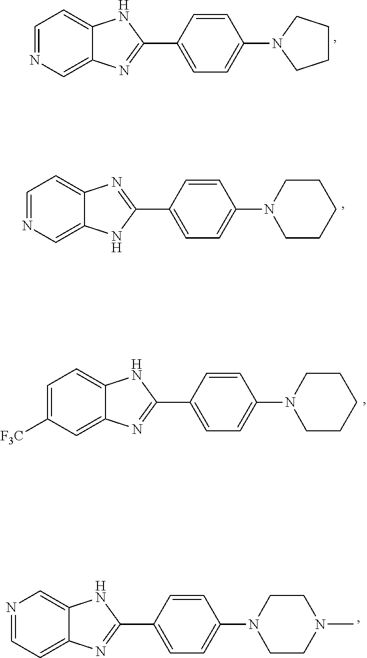 [SMILES: c1cc2[nH]c(-c3ccc(N4CCCC4)cc3)nc2cn1 ; c1cc2nc(-c3ccc(N4CCCCC4)cc3)[nH]c2cn1 ; FC(F)(F)c1ccc2[nH]c(-c3ccc(N4CCCCC4)cc3)nc2c1 ; CN1CCN(c2ccc(-c3nc4ccncc4[nH]3)cc2)CC1]
or a pharmaceutically acceptable salt thereof.

26. A method of treating a disorder associated with Wnt dysfunction in a subject, the method comprising administering to the subject an effective amount of at least one compound having a structure represented by a formula: 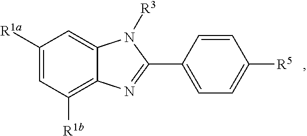
wherein each of R^{1a} and R^{1b} is independently selected from halogen, —CF₃, and —OR²⁰;
wherein each occurrence of R²⁰, when present, is independently selected from hydrogen, C1-C4 alkyl, C1-C4 monohaloalkyl, and C1-C4 polyhaloalkyl;
wherein R³ is selected from hydrogen and C1-C4 alkyl; and
wherein R⁵ is selected from halogen, —OH, and —CF₃,
or a pharmaceutically acceptable salt thereof.

27. The method of claim 26, wherein the subject has been diagnosed with the disorder prior to the administering step.

28. The method of claim 28, wherein the compound is selected to have activity of less than about 5 μM against a pancreatic cancer stem cell line.

29. The method of claim 28, wherein the pancreatic cancer stem cell line is selected from PANC-1, Suit-2, S2VP10, and L3.6p1.

30. A method of treating a disorder associated with Wnt dysfunction in a subject, the method comprising administering to the subject an effective amount of at least one compound having a structure represented by a formula: 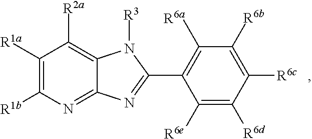
wherein each of R^{1a} and R^{1b} is independently selected from halogen, —CF₃, and —OR²⁰;
wherein each occurrence of R²⁰, when present, is independently selected from hydrogen, C1-C4 alkyl, C1-C4 monohaloalkyl, and C1-C4 polyhaloalkyl;
wherein R^{2a} is selected from hydrogen, halogen, —CN, —OH, —SH, —NH₂, C1-C4 alkyl, C1-C4 monohaloalkyl, C1-C4 polyhaloalkyl, C1-C4 alkoxy, C1-C4 thioalkoxy, C1-C4 monohaloalkoxy, and C1-C4 polyhaloalkoxy;
wherein R³ is selected from hydrogen and C1-C4 alkyl; and
wherein each of R^{6a}, R^{6b}, R^{6c}, R^{6d}, and R^{6e} is independently selected from hydrogen, halogen, —CF₃, and —OR²⁰,
or a pharmaceutically acceptable salt thereof.

31. The method of claim 30, wherein the subject has been diagnosed with the disorder prior to the administering step.

32. The method of claim 30, wherein the compound is selected to have activity of less than about 5 μM against a pancreatic cancer stem cell line.

33. The method of claim 32, wherein the pancreatic cancer stem cell line is selected from PANC-1, Suit-2, S2VP10, and L3.6p1.
